(19) United States
(12) Patent Application Publication
(10) Pub. No.: US 2024/0339175 A1
(43) Pub. Date: Oct. 10, 2024

(54) OBJECT DETERMINING METHOD AND APPARATUS, COMPUTER DEVICE, AND STORAGE MEDIUM

(71) Applicant: Tencent Technology (Shenzhen) Company Limited, Shenzhen (CN)

(72) Inventors: Lixue CHENG, Shenzhen (CN); Ziyi YANG, Shenzhen (CN); Benben LIAO, Shenzhen (CN); Shengyu ZHANG, Shenzhen (CN)

(21) Appl. No.: 18/745,916

(22) Filed: Jun. 17, 2024

Related U.S. Application Data
(63) Continuation of application No. PCT/CN2023/084640, filed on Mar. 29, 2023.

(30) Foreign Application Priority Data
May 9, 2022 (CN) 202210498684.7

(51) Int. Cl. G16B 20/50; G16B 35/20; G16B 40/00

(52) U.S. Cl. CPC G16B 20/50; G16B 35/20; G16B 40/00

(57) ABSTRACT

Provided is an object determining method performed by a computer device, relating to the technical field of artificial intelligence. The method includes: acquiring index prediction values of objects in a first object set on a preset index respectively; determining, based on index experimental values and object features of the objects in the first object set on the preset index, a mapping relationship between the preset index and the object features; selecting, from the first object set, objects with the index prediction values satisfying index value screening conditions to obtain a second object set; and determining a target object meeting index requirements of the preset index from the second object set based on the mapping relationship.

### CROSS-REFERENCE TO RELATED APPLICATIONS

This application is a continuation application of PCT Patent Application No. PCT/CN2023/084640, entitled “OBJECT DETERMINING METHOD AND APPARATUS, COMPUTER DEVICE, AND STORAGE MEDIUM” filed on Mar. 29, 2023, which claims priority to Chinese Patent Application No. 202210498684.7, entitled “OBJECT DETERMINING METHOD AND APPARATUS, COMPUTER DEVICE, AND STORAGE MEDIUM” filed to the China National Intellectual Property Administration on May 9, 2022, all of which is incorporated by reference in its entirety.

### FIELD OF THE TECHNOLOGY

This application relates to the field of computer technologies, and in particular, to an object determining method and apparatus, a computer device, and a storage medium.

### BACKGROUND OF THE DISCLOSURE

With the development of computer technology, directed evolution has emerged. The directed evolution may obtain proteins with new functions and characteristics in a short time. By setting clear goals, molecules may be redesigned. The directed evolution has become an important research tool in the fields of new drug research and chemical engineering.

In the traditional directed evolution of proteins, an initial protein is established for a target function. A mutant library is constructed at one or more positions. Most common mutants are determined by screening, and these mutants are randomly recombined and screened. A next round of “mutation, recombination and screening” is performed by using the screened mutants until an expected protein performance is achieved.

However, most of the current directed evolution technologies are laborious and time-consuming, and the time cost is high.

### SUMMARY

According to various embodiments provided in this application, an object determining method and apparatus, a computer device, a computer-readable storage medium, and a computer program product are provided.

According to one aspect, this application provides an object determining method performed by a computer device. The method includes: acquiring index prediction values of objects in a first object set on a preset index respectively; determining, based on index experimental values and object features of the objects in the first object set on the preset index, a mapping relationship between the preset index and the object features; selecting, from the first object set, objects with the index prediction value satisfying index value screening conditions, to obtain a second object set; and determining a target object meeting index requirements of the preset index from the second object set based on the mapping relationship.

According to another aspect, this application further provides a computer device. The computer device includes a memory and one or more processors. The memory stores computer-readable instructions. The computer-readable instructions, when executed by the processor, enable the computer device to perform the steps of the object determining method.

According to another aspect, this application further provides one or more non-transitory readable storage media. The computer-readable storage medium stores computer-readable instructions. The computer-readable instructions, when executed by one or more processors of a computer device, enable the computer device to perform the steps of the object determining method.

Details of one or more embodiments of this application are provided in the accompanying drawings and descriptions below. Other features, objectives, and advantages of this application become apparent from the specification, the drawings, and the claims.

### BRIEF DESCRIPTION OF THE DRAWINGS

To describe the technical solutions of the embodiments of this application more clearly, the following briefly introduces the accompanying drawings required for describing the embodiments. Apparently, the accompanying drawings in the following description show only some embodiments of this application, and those of ordinary skill in the art may still derive other drawings from these accompanying drawings without creative efforts.

FIG. 1 is a diagram of an application environment of an object determining method in some embodiments.

FIG. 2 is a schematic flowchart of an object determining method in some embodiments.

FIG. 3A is an application diagram of enzyme in some embodiments.

FIG. 3B is a schematic diagram of machine learning assisted directed evolution in some embodiments.

FIG. 4 is a schematic diagram of a mean fitness of amino acids in some embodiments.

FIG. 5 is a schematic flowchart of an object determining method in some embodiments.

FIG. 6 is a schematic diagram of an object determining method in some embodiments.

FIG. 7 is a schematic diagram of an object determining method in some embodiments.

FIG. 8 is a diagram of an application environment of an object determining method in some embodiments.

FIG. 9 is a diagram of an application environment of an object determining method in some embodiments.

FIG. 10 is a fitness distribution diagram of different datasets in some embodiments.

FIG. 11 is an effect diagram of different methods on four protein-directed evolution datasets in some embodiments.

FIG. 12 is an effect diagram of different methods on a dataset in some embodiments.

FIG. 13 is a structural block diagram of an object determining apparatus in some embodiments.

FIG. 14 is an internal structure diagram of a computer device in some embodiments.

FIG. 15 is an internal structure diagram of a computer device in some embodiments.

### DESCRIPTION OF EMBODIMENTS

To make the objects, technical solutions, and advantages of this application clearer, the following further describes this application in detail with reference to the accompanying drawings and the embodiments. It is to be understood that specific embodiments described herein are merely illustrative of this application and are not intended to be limiting thereof.

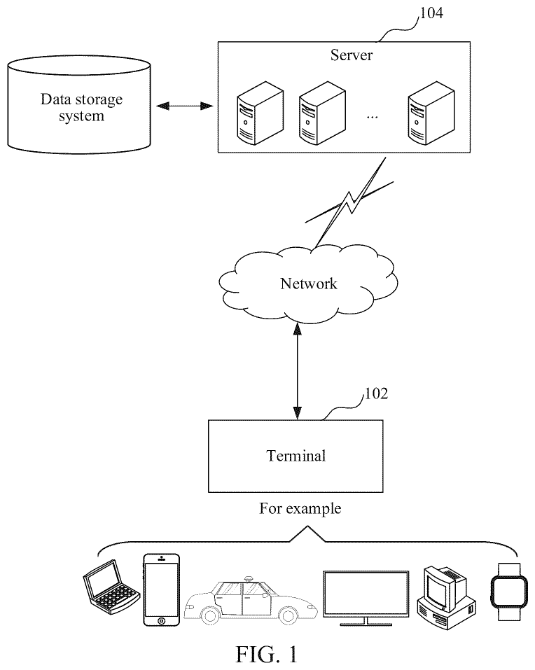

An object determining method provided in embodiments of this application may be applied to an application environment as shown in FIG. 1. A terminal 102 communicates with a server 104 via a network. A data storage system may store data to be processed by the server 104. The data storage system may be integrated on the server 104 or may be placed on a cloud or other servers.

Specifically, the server 104 may acquire index prediction values of objects in a first object set on a preset index respectively, select an object with the index prediction value satisfying index value screening conditions from the first object set to obtain a second object set, determine, based on index experimental values and object features of the plurality of objects in the first object set on the preset index, a mapping relationship between the preset index and the object features, and determine a target object meeting index requirements of the preset index from the second object set based on the mapping relationship. After determining the target object, the server 104 may store the target object and transmit the target object to the terminal 102. The terminal 102 may display relevant information of the target object.

The terminal 102 may be, but is not limited to, various desktop computers, notebook computers, smart phones, tablet computers, Internet of Things devices, and portable wearable devices. The Internet of Things devices may be smart speakers, smart televisions, smart air conditioners, smart vehicle-mounted devices, or the like. The portable wearable devices may be smart watches, smart bracelets, head-mounted devices, or the like. The server 104 may be implemented as a stand-alone server or as a server cluster composed of a plurality of servers.

In some embodiments, the index prediction values may be predicted by a trained index detection model. The index detection model may be based on artificial intelligence and machine learning. For example, the index detection model may be a neural network model.

The scheme provided by this embodiment of this application relates to an artificial intelligence neural network and other technologies, and is specifically described by the following embodiments.

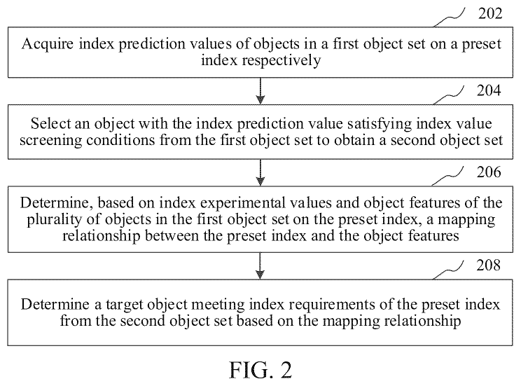

In some embodiments, as shown in FIG. 2, an object determining method is provided. The method may be performed by a terminal or a server, and may also be performed jointly by the terminal and the server. The method, illustrated by being applied to the server 104 in FIG. 1, includes the following steps:

Step 202: Acquire index prediction values of objects in a first object set on a preset index respectively.

A plurality of objects are included in the first object set. The object may be a true substance including but not limited to at least one of a protein, a material, or a battery. The object may also be an abstract concrete concept. For example, the object is a battery fast charging protocol.

The object may correspond to a plurality of indexes, and the preset index may be any one of a plurality of indexes of the object. For example, if the object is a protein, the index of the object includes, but is not limited to, at least one of fitness, enrichment score, activity or brightness. If the object is a material, the index of the object includes, but is not limited to, at least one of components of the material or the proportion of components. If the object is a battery fast charging protocol, the index of the object includes, but is not limited to, various parameters of the battery fast charging protocol.

The index prediction values are values corresponding to the preset index, which are predicted by the objects. The index prediction values are values of objects predicted by a pre-trained model on the preset index. The model has the function of predicting the values of the objects on the preset index. The index prediction values may be predicted by a trained index detection model. The index detection model may be a neural network model.

The objects in the first object set may belong to the same object class, which includes but is not limited to at least one of substances such as proteins or materials, and may also include abstract concepts such as a battery charging protocol. For example, the objects in the first object set belong to a certain kind of protein. For example, the objects are mutant proteins obtained by mutating the same protein. The mutant proteins are relative to wild-type proteins. The wild-type proteins are unmutated proteins. The mutant proteins are proteins obtained after mutation on the basis of the wild-type proteins. In the directed evolution of proteins, the required proteins may be obtained by mutation. There are two mutation scenarios in the directed evolution of proteins, including a k-site saturated mutagenesis scenario and an unsaturated mutagenesis scenario.

The k-site saturated mutagenesis scenario is adapted to mutate amino acids at k designated mutation sites. In the mutant protein generated under the scenario, amino acids on at least one of the k designated mutation sites are obtained by mutation. For example, k=4. Then the amino acids on at least one of the four designated mutation sites in the obtained mutant protein are obtained by mutation. That is, the position and number of mutation sites in the k-site saturated mutagenesis scenario are fixed, and mutation only occurs at the k designated mutation sites. The mutation site refers to a position in a protein where mutation may occur. Therefore, the mutation site may also be referred to as a mutation position. There is an amino acid at each position in the protein.

In the unsaturated mutagenesis scenario, the mutation site is not fixed but the number of mutated amino acids is fixed. For example, in each mutant protein obtained in the unsaturated mutagenesis scenario, amino acids at two positions are obtained by mutation, but the mutation positions may be the same or different. For example, mutation occurs in position 1 and 2, and another mutation occurs in position 3 and 4.

If the object class is a protein, the objects in the first object set may be mutant proteins generated in the k-site saturated mutagenesis scenario or mutant proteins generated in the unsaturated mutagenesis scenario. The mutant proteins may also be referred to as mutants. The proteins may be represented by amino acid sequences. If the first object set includes n mutant proteins, the first object set may be represented as {Sᵢ, yᵢ}ᵢ₌₁ⁿ, where n represents the number of mutants, Sᵢ represents mutants, Sᵢ=(Sᵢ₁, Sᵢ₂, . . . , S_{iL}), Sᵢ represents an i^{th} amino acid sequence having L amino acids, Sᵢⱼ represents amino acids, 1≤j≤L, and yᵢ represents the fitness of an i^{th} protein. The fitness is obtained by experimental measurement. The fitness of the protein represents the characteristics of the protein, and the fitness may be, for example, affinity.

Specifically, for each object in the first object set, the server may predict the index prediction value of each object on the preset index to obtain the index prediction value of each object. For example, the index prediction value may be predicted by using the trained index detection model.

In some embodiments, the server may screen a plurality of objects from the first object set to obtain a reference object set, determine an object feature, namely a feature of object, of each object in the reference object set, and experimentally acquire a value of each object in the reference object set on the preset index to obtain an index experimental value of each object in the reference object set. The index experimental value refers to a value of the object on the preset index acquired experimentally. That is, the index experimental value of the object is a true value of the object on the preset index. For example, if the object is a protein and the preset index is fitness, the index experimental value refers to a fitness value of the protein acquired experimentally. The server may train the index detection model to obtain a trained index detection model by using the object features of the objects in the reference object set and the index experimental values of the objects, determine object features of the objects in the first object set, input the object features of the objects in the first object set into the trained index detection model, and predict an index prediction value corresponding to each object in the first object set by using the trained index detection model.

In some embodiments, the object is a mutant protein, the object feature is a protein feature, and the protein feature may be a feature encoded based on an amino acid at a mutation site in the mutant protein. For example, the amino acid may be encoded based on the index experimental value of the mutant protein on the preset index to obtain an amino acid feature corresponding to the amino acid, and a protein feature of the mutant protein may be obtained based on the amino acid feature corresponding to the amino acid at each mutation position. For example, for a mutant protein generated in the k-site saturated mutagenesis scenario, the protein feature of the mutant protein may be obtained by using the amino acid feature of the amino acid at the k-site. For a mutant protein generated in the unsaturated mutagenesis scenario, if there are mutations at two positions, a vector composed of amino acid features of amino acids at the two positions is determined as the protein feature of the mutant protein.

Step 204: Select an object with the index prediction value satisfying index value screening conditions from the first object set to obtain a second object set.

The index value screening conditions include that the index prediction value is greater than a first index threshold. The first index threshold may be preset or set as required. The second object set is a set composed of objects screened from the first object set. The index prediction values of objects in the second object set satisfy the index value screening conditions.

Specifically, the server may compare the index prediction value of each object in the first object set with the first index threshold, and combine the objects with the index prediction values greater than the first index threshold into the second object set. For example, if the object is a mutant protein, the preset index is affinity and the first index threshold is an affinity threshold, the objects with the affinity greater than the affinity threshold in the first object set are combined into the second object set. The affinity threshold may be preset or set as required.

Step 206: Determine, based on index experimental values and object features of the plurality of objects in the first object set on the preset index, a mapping relationship between the preset index and the object features.

The plurality of objects in the first object set may refer to the objects in the reference object set. The mapping relationship between the preset index and the object features is used for reflecting the change of the value of the preset index with the change of the object features. The mapping relationship between the preset index and the object features may be represented by a curve. For example, the mapping relationship may be represented by a curve y1=f1(x), where y1 represents the preset index, and x represents the object features.

Specifically, after obtaining the reference object set, the server may train the index detection model by using the reference object set, and also determine a mapping relationship between the preset index and the object features by using the index experimental values and the object features of the objects in the reference object set on the preset index.

In some embodiments, after obtaining the index experimental values of the objects in the reference object set, the server may use, for each object in the reference object set, the object feature and the index experimental value of the object as points on the curve y1=f1(x), and a plurality of points on a plurality of curves are fitted to generate the curve y1=f1(x) representing the mapping relationship.

Step 208: Determine a target object meeting index requirements of the preset index from the second object set based on the mapping relationship.

The index requirements of the preset index may, for example, be at least one of the index experimental value being as large as possible or the index experimental value being greater than a second index threshold. The target object is an object in the second object set that meets the index requirements of the preset index.

Specifically, the mapping relationship between the preset index and the object features is a first mapping relationship. The server may perform statistical operation based on the first mapping relationship to obtain a second mapping relationship between a target statistical index and the object features, determine a statistical index value of each object in the second object set on the target statistical index based on the second mapping relationship, determine a selected object from the objects in the second object set based on the statistical index value of each object, and obtain an object meeting the index requirements of the preset index based on the selected object. The first mapping relationship represents the rule that the value of the preset index changes with the change of the object features. The second mapping relationship represents the rule that the value of the target statistical index changes with the change of the object features. For example, the second mapping relationship may be represented by a curve y2=f2(x), where y2 represents the target statistical index, and x represents the object features.

There may be one or more target statistical indexes. For example, the target statistical index includes, but is not limited to, at least one of expected improvement (EI), probability of improvement (PI), upper confidence bound (UCB), or Thompson sampling (TS). The first mapping relationship may also be referred to as a probability surrogate model. The second mapping relation relationship may also be referred to as a collection function. The collection function is constructed by a posterior probability distribution obtained by the probability surrogate model, and a next most “potential” experimental point is selected by maximizing the collection function. The collection function is responsible for testing the proposed new points based on the trade-off between exploration and utilization. The exploration is to select a point far away from a known point for a next experiment, namely, to explore an unknown area. The utilization is to select a point close to the known point for the next experiment, namely, to dig points around the known point.

In some embodiments, the server may determine a statistical index value of each object in the second object set on the target statistical index based on the second mapping relationship and determine a selected object from the objects in the second object set based on the statistical index value of each object. Specifically, there may be one or more selected objects. The object corresponding to the largest statistical index value may be determined as the selected object. Alternatively, the object with the statistical index value greater than a third index threshold may be determined as the selected object, and the third index threshold may be set as required. The server may obtain an object meeting the index requirements of the preset index based on the selected object. For example, the server may determine the selected object as the object meeting the index requirements of the preset index.

In some embodiments, after obtaining the selected object, the server may acquire the index experimental value of the selected object, and compare the index experimental value of the selected object with the second index threshold. When it is determined that the index experimental value of the selected object reaches the second index threshold, the selected object is determined as a target object. After the selected object is obtained, the corresponding index experimental value may be determined experimentally. If it is determined that the index experimental value of the selected object does not reach the second index threshold, the selected object may be added to the reference object set, and the first mapping relationship between the preset index and the object features is determined again by using the index experimental values and the object features of the objects in the reference object set, thereby determining the target object meeting the index requirements of the preset index from the second object set again based on the first mapping relationship. After continuous cycles, when the index experimental value reaches the second index threshold, the selected object is determined as the target object, or when the cycles reach a number threshold, the selected object is determined as the target object.

In the object determining method, index prediction values of objects in a first object set on a preset index respectively are acquired. An object with the index prediction value satisfying index value screening conditions is selected from the first object set to obtain a second object set. Based on index experimental values and object features of the plurality of objects in the first object set on the preset index, a mapping relationship between the preset index and the object features is determined. A target object meeting index requirements of the preset index is determined from the second object set based on the mapping relationship. Since the second object set is screened from the first object set, it is more efficient to determine the target object meeting the index requirements of the preset index from the second object set than to screen the target object from the first object set, thus reducing the time cost for determining the target object.

In practical design application scenarios, environmentalists acquire environmental conditions by designing sensor deployment positions. Chemists acquire new substances by designing experiments. Pharmaceutical manufacturers design new drugs to resist diseases. Generally, these design problems are considered as the following optimization problems to be solved (only the maximization problem is considered, and the minimization problem may be simply transformed into the minimization problem by taking a minus sign operation):

$${x}^{*} = arg {max f}_{x ∈ X ⊆ {ℝ}^{d}} ( x )$$

where x represents a d-dimensional decision vector, X represents a decision space, and f (x) represents an objective function. Corresponding to the above examples, x may be represented as the sensor deployment position, an experimental configuration, a drug formulation, and the like, and f (x) may be represented as a measure of the performance of environment, experiment, formulation, and the like. In these practical design application scenarios, there are many complex design decisions. Optimization objectives usually have the following characteristics: high calculation cost: ideally, the function may be executed many times to determine an optimal solution, but it is unrealistic to calculate over-sampling in practical optimization problems, and the calculation cost is very high; black box function: in practical problems, the structure of the objective function is difficult to describe mathematically, and there is no first-order or higher-order derivative, which cannot be solved by gradient descent or Newton related algorithm; to find a global minimum/maximum: a certain mechanism is needed to avoid falling into a local minimum/maximum. Therefore, in order to acquire required substances, a high time cost is needed.

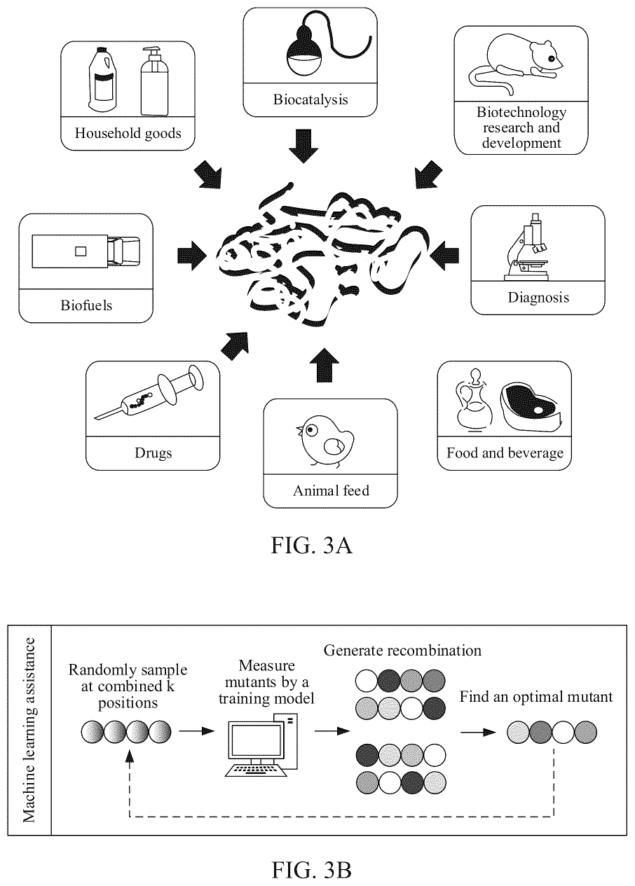

The object determining method provided by this application can speed up the process of acquire a required object, improve the efficiency, and reduce the time cost. For example, the object determining method provided by this application may be applied to computing method assisted protein evolution to obtain required proteins. Proteins play an important role in people's lives. For example, enzymes play an important role in human society, from daily to industrial, as shown in FIG. 3A. Some washing powders used daily contain enzymes to promote the decomposition of stains such as oil stains. In the process of fermentation and degradation in food industry, enzymes are essential. As green and efficient catalysts, enzymes in drugs and fine chemicals have replaced some production processes that require heavy metals and consume high energy in traditional chemistry. Furthermore, enzymes are the most important role in the development of biological energy. Directed evolution may obtain proteins with new functions and characteristics in a short time. By manually setting clear goals, molecules may be redesigned by scientists. The directed evolution has become an important research tool in the fields of new drug research and chemical engineering. The directed evolution may be assisted by machine learning. As shown in FIG. 3B, for example, the process of machine learning assisted directed evolution may include four steps: 1) establishing initial proteins for target functions, and constructing mutant libraries at k positions; 2) training a model by using existing data; 3) predicting other mutants in the mutant libraries by using the trained model; and 4) selecting the best mutant for experimental test, and adding the mutant to a training set for a next round of model training. The computing method assisted directed evolution can speed up optimization and reduce experimental burden.

In some embodiments, the objects in the first object set are mutant proteins. The method further includes: screening based on the first object set to obtain a reference object set, the reference object set satisfying a condition that each amino acid occurs at each mutation position for at least a target frequency. The acquiring index prediction values of objects in a first object set on a preset index respectively includes: training an index detection model based on an object feature and an index experimental value of each object in the reference object set; and predicting the index prediction value of each object in the first object set by using the trained index detection model.

The objects in the reference object set may be mutant proteins, or the reference object set may include wild-type proteins and mutant proteins. The objects in the first object set are mutant proteins. The target frequency may be preset or set as required, for example, may be 2. The mutation position is a mutation site. The reference object set satisfies a condition that each amino acid occurs at each mutation position for at least a target frequency. For example, if there are 20 amino acids and the target frequency is 2, the 20 amino acids occur at each mutation site at least twice in the proteins in the reference object set. For a mutant protein generated by the k-site saturated mutagenesis scenario, there are 4 mutation sites, and each of 20 amino acids occur at each mutation site twice. Then 40 samples may be selected from a sample space as initial samples. In this way, the maximum coverage of amino acid encoding information covered in an initial sample size is ensured, the least experiments are required, and the experimental cost is reduced. The sample space may include mutant proteins and wild-type proteins. The samples refer to proteins. The reference object set may be constantly changing. The initial samples refer to the initially determined reference object set.

The index detection model is configured to determine a value of an object on the preset index according to the object feature, that is, to determine an index prediction value of the object on the preset index. The index detection model may be a neural network model, for example, improving supervised outlier detection with unsupervised representation learning (XGBOD), may definitely be another model, and will not be limited herein. The basic process of XGBOD is to learn original data by using a variety of unsupervised models, obtain outlier scores of the samples, and take the outlier scores as a new data representation. Then, original features are merged to generate a new feature space. Finally, an XGBoost classifier is trained in the new feature space, and outputs a prediction result.

Specifically, the server may acquire an index experimental value of each object (denoted as the index experimental value corresponding to the object) in the reference object set on the preset index, and determine an object feature of each object in the reference object set based on the index experimental value of each object in the reference object set on the preset index. The server may input the object feature of the object into a to-be-trained index detection model for prediction to obtain the index prediction value of the object (denoted as the index experimental value corresponding to the object) on the preset index, and adjust model parameters of the index detection model based on a difference between the index experimental value corresponding to the object and the corresponding index prediction value until the model converges, so as to obtain a trained index detection model. The server may input the object feature of each object in the first object set into the trained index detection model for prediction, so as to obtain the index prediction value corresponding to each object in the first object set.

In some embodiments, the server may determine an amino acid feature of each amino acid based on the index experimental value of each object in the reference object set. Therefore, when determining the object feature (namely, protein feature) of the object in the first object set, the server may determine the protein feature of the object in the first object set by using the amino acid feature of each amino acid determined by the reference object set. For example, in the k-site saturated mutagenesis scenario, the amino acid feature of each amino acid at each mutation position may be determined based on the index experimental value of each object in the reference object set. For an object in the first object set, amino acids at mutation positions in the object may be determined. The amino acid feature corresponding to the amino acid at each mutation position in the object may be determined from the determined “amino acid feature of each amino acid at each mutation position”. A vector composed of the determined amino acid features is determined as the object feature (namely, protein feature) of the object.

In some embodiments, the method for screening based on the first object set to obtain a reference object set may be used as a sample selection policy for determining an initial sample in Bayesian optimization, thereby improving optimization efficiency, and reducing the time cost of Bayesian optimization. The maximum coverage of amino acid encoding information covered in an initial sample size is ensured, the least experiments are required, and the experimental cost is reduced. The Bayesian optimization usually uses Gaussian process (GP) regression based on Gaussian distribution as a prior probability surrogate model. GP is flexible and scalable, and can represent any linear/nonlinear function in theory. Gaussian process regression based on student-t prior may definitely be used as the prior probability surrogate model, and robust regression (Gaussian process based on student-t distribution) may be combined with outlier detection. Data points are divided into outliers and inliers, so as to eliminate the influence of outliers on model fitting. The Gaussian process based on student-t prior may be referred to as “Robust GP” for short, and the Gaussian process based on Gaussian distribution may be referred to as “GP” for short.

In this embodiment, since the reference object set satisfies the condition that each amino acid occurs at each mutation position for at least the target frequency, the number of amino acids in the obtained reference object set is balanced. Therefore, the index detection model is trained based on the reference object set, so as to improve the training accuracy, thereby improving the accuracy of the index prediction value predicted by the trained index detection model.

In some embodiments, the determining, based on index experimental values and object features of the plurality of objects in the first object set on the preset index, a mapping relationship between the preset index and the object features includes: determining the object features of the objects in the reference object set based on the index experimental value of each object in the reference object set on the preset index; and determining a mapping relationship between the preset index and the object features based on the index experimental value and the object feature of each object in the reference object set on the preset index.

Specifically, the server may statistically calculate the index experimental value corresponding to each object in the reference object set to obtain the object features of the objects in the reference object set. The index experimental value corresponding to the object refers to the index experimental value of the object on the preset index. The statistical calculation includes, but is not limited to, calculating at least one of a mean, a maximum, or a minimum.

In some embodiments, the mapping relationship between the preset index and the object features is a first mapping relationship, and the mapping relationship may be represented by a curve. For example, the first mapping relationship is represented by a curve y1=f1(x). The server may use, for each object in the reference object set, the object feature and the index experimental value of the object as points on the curve y1=f1 (x), and a plurality of points on a plurality of curves are fitted to generate the curve y1=f1 (x) representing the mapping relationship.

In this embodiment, since the reference object set satisfies the condition that each amino acid occurs at each mutation position for at least the target frequency, the amino acids in the obtained reference object set is balanced. Therefore, the object features of the objects in the reference object set are determined based on the index experimental value of each object in the reference object set on the preset index, so that the coverage range of amino acid encoding information is larger, that is, an information range covered by the object features is improved.

In some embodiments, the screening based on the first object set to obtain a reference object set includes: acquiring a current score set, the current score set including a current score corresponding to each amino acid; obtaining a second protein set based on the first object set, and selecting a target protein from the second protein set based on the current score set; decreasing a current score corresponding to an amino acid at each mutation position in the target protein in the current score set, and moving the target protein from the second protein set to a first protein set; and reselecting, when the current score set characterizes the first protein set as not satisfying the condition that each amino acid occurs at each mutation position for at least the target frequency, a target protein from the second protein set based on the current score set until the current score set characterizes the first protein set as satisfying the condition that each amino acid occurs at each mutation position for at least the target frequency, and determining the first protein set as the reference object set.

The current score set includes a current score corresponding to each amino acid. The current score is an integer, for example, 2. The current score corresponding to each amino acid may be different or the same. Each amino acid may correspond to one or more current scores. For example, the same amino acid corresponds to the current score at different mutation positions. The current score set is constantly changing. The target protein is selected from the second protein set based on the current score set. There may be one or more target proteins.

Specifically, the server may determine the first object set as the second protein set. That is, the objects in the second protein set are consistent with the objects in the first object set. Alternatively, the server may acquire objects other than the objects in the reference object set from the first object set to form the second object set.

In some embodiments, a current protein score for each mutant protein in the second protein set is determined from the current score set, and the mutant protein corresponding to the maximum current protein score is determined as the target protein. The server may arrange the mutant proteins in the second protein set in descending order of current protein scores to obtain a first protein sequence, and determine the mutant protein in the first protein sequence which is arranged ahead of a sorting threshold as the target protein. The sorting threshold may be preset or set as required, for example, any one of 1^{st} or 2^{nd}.

In some embodiments, the server may decrease a current score corresponding to an amino acid at each mutation position in the target protein in the current score set, and move the target protein from the second protein set to the first protein set.

For example, if the object in the first object set is a mutant protein generated in the k-site saturated mutagenesis scenario, the current score set includes the current score of each amino acid at each mutation position. If the number of mutation positions is 4, the current score set may be represented in a matrix form, and the current score set may also be referred to as a current score matrix. In the matrix corresponding to the current score set, (row u, column w) represents a current score corresponding to a u^{th} amino acid at a w^{th} mutation position, 1≤u≤m, and 1≤w≤k, where m is the number of amino acid types, for example, 20, and k represents the number of mutation positions, for example, 4. For example, if the amino acid at a first mutation position in the mutant protein is a first amino acid, the corresponding current score is a score in (row 1, column 1) in the matrix.

If the object in the first object set is a mutant protein generated in the unsaturated mutagenesis scenario, the current score set includes a current score corresponding to each amino acid. The current score set may be represented by a vector. The current score set may also be referred to as a current score vector. The score arranged at a u^{th} position in the vector represents a current score of a u^{th} amino acid. For example, if the amino acid at the first mutation position in the mutant protein is the first amino acid, the corresponding current score is a top score in the vector.

In some embodiments, the server reselects, when the current score set characterizes the first protein set as not satisfying the condition that each amino acid occurs at each mutation position for at least the target frequency, a target protein from the second protein set based on the current score set until the current score set characterizes the first protein set as satisfying the condition that each amino acid occurs at each mutation position for at least the target frequency, and determines the first protein set as the reference object set. Specifically, the first protein set is constantly changing. If the initial first protein set does not include any protein, the scores in the initial current score set are equal to the target frequency. For example, if the target frequency is 2, each current score in the current score set is equal to 2, and the score in the initial current score set is equal to 2. After determining a target protein, the target protein may be moved from the second protein set to the first protein set, and current scores corresponding to amino acids at mutation positions in the target protein in the current score matrix are decreased by 1 each time, for example, from 2 to 1 or from 1 to 0, thereby continuously updating the current score matrix. When there is no score greater than 0 in the current score matrix, it is determined that the first protein set satisfies the condition that each amino acid occurs at each mutation position for at least the target frequency, thereby determining the first protein set as the reference object set.

In this embodiment, the second protein set is obtained based on the first object set, the target protein is selected from the second protein set, the current score set is updated based on the target protein, and the target protein is moved from the second protein set to the first protein set to continuously select proteins to update the current score. When the current score set characterizes that the first protein set satisfies the condition that each amino acid occurs at each mutation position for at least the target frequency, the first protein set is determined as the reference object set. Therefore, the reference object set satisfying one of the conditions that each amino acid occurs at each mutation position for at least the target frequency is quickly selected from the first object set, and the experiments for determining the reference object set are reduced, thereby reducing the experimental cost and the time cost.

In some embodiments, the acquiring a current score set includes: acquiring an initial score set, an initial score corresponding to each amino acid in the initial score set being the target frequency; and decreasing an initial score corresponding to an amino acid at each mutation position in a wild-type protein, respectively, from the initial score set to obtain the current score set, and determining the first protein set based on the wild-type protein, the wild-type protein being an unmutated protein.

The initial score corresponding to each amino acid in the initial score set is the target frequency. If the target frequency is an integer, for example, 2, the initial score is 2. For example, if the object in the first object set is a mutant protein generated in the k-site saturated mutagenesis scenario, the initial score set includes the initial score of each amino acid at each mutation position. If the number of mutation positions is 4, the initial score set may be represented in a matrix form, and each element in the matrix is 2. In the matrix corresponding to the initial score set, (row u, column w) represents an initial score corresponding to a u^{th} amino acid at a w^{th} mutation position, 1≤u≤m, and 1≤w≤k, where m is the number of amino acid types, for example, 20, and k represents the number of mutation positions, for example, 4. For example, if the amino acid at a first mutation position in the mutant protein is a first amino acid, the corresponding initial score is an initial score in (row 1, column 1) in the matrix.

If the object in the first object set is a mutant protein generated in the unsaturated mutagenesis scenario, the initial score set includes an initial score corresponding to each amino acid. The initial score set may be represented by a vector, and elements in the vector are 2. The score arranged at a u^{th} position in the vector represents an initial score of a u^{th} amino acid. If the amino acid at a first mutation position in the mutant protein is a first amino acid, the corresponding initial score is a top score in the vector.

Specifically, in the k-site saturated mutagenesis scenario, the server may determine the amino acid corresponding to each mutation position from the wild-type protein, and decrease the current score corresponding to the amino acid in the initial score set by 1 each time to obtain the current score set. In a 4-site saturated mutagenesis scenario, the number of mutation positions is 4. If amino acids corresponding to the four mutation positions in the wild-type protein are a first amino acid A1, a second amino acid A2, a third amino acid A3, and a fourth amino acid A4 respectively, scores in (row 1, column 1), (row 2, column 2), (row 3, column 3), and (row 4, column 4) in the matrix corresponding to the initial score set are all reduced by 1, and the initial score set with the scores reduced by 1 is determined as the current score set.

In the unsaturated mutation scenario, each mutant protein corresponds to a target number of mutation positions, and the target number is for example 2. The server may count the mutation positions corresponding to a plurality of mutant proteins in the unsaturated mutation scenario to obtain a mutation position set. The server may determine an amino acid corresponding to each mutation position in the mutation position set from the wild-type protein. The server may determine the amino acid corresponding to each mutation position in the wild-type protein from the wild-type protein, and decrease a current score corresponding to each determined amino acid in the initial score set by 1 each time to obtain the current score set.

In some embodiments, the server may determine a set of wild-type proteins as the first protein set. That is, the initial first protein set includes a wild-type protein.

In this embodiment, the initial score set is updated based on the unmutated wild-type protein to obtain the current score set, thereby increasing the speed of score decrease, improving the efficiency of obtaining the reference object set, and reducing the time cost.

In some embodiments, the selecting a target protein from the second protein set based on the current score set includes: determining, for each mutant protein in the second protein set, a current score corresponding to an amino acid at each mutation position in the mutant protein, respectively, from the current score set; determining a current protein score of the mutant protein based on the obtained current scores; and selecting the target protein from the second protein set based on the current protein score.

Specifically, for each mutant protein in the second protein set, a current score corresponding to an amino acid at each mutation position in the mutant protein, respectively, is determined from the current score set, the obtained current scores are summed, and the result of summation calculation is determined as the current protein score of the mutant protein.

In some embodiments, the server may arrange the mutant proteins in the second protein set in descending order of current protein scores to obtain a first protein sequence, and determine the mutant protein in the first protein sequence which is arranged ahead of a sorting threshold as the target protein. The sorting threshold may be preset or set as required, for example, any one of 1^{st} or 2^{nd}.

In this embodiment, a mutant protein with a larger current protein score is selected from the second protein set to obtain a target protein. As the current protein score is larger, the strength of updating the current score set is larger, thereby increasing the speed of making the current score set characterize that the first protein set satisfies the condition that each amino acid occurs at each mutation position for at least the target frequency, and improving the efficiency of obtaining the reference object set.

In some embodiments, each amino acid corresponds to an amino acid, and the score in the current score set is uniquely identified by the amino acid and the mutation position. The determining a current score corresponding to an amino acid at each mutation position in the mutant protein, respectively, from the current score set includes: determining, for the amino acid at each mutation position, a current score corresponding to the amino acid at the mutation position from the current score set according to the amino acid corresponding to the amino acid and the mutation position.

Specifically, when the object in the first object set is a mutant protein generated in the k-site saturated mutagenesis scenario, the current score set includes the current score of each amino acid at each mutation position. That is, the score in the current score matrix is uniquely identified by the amino acid and the mutation position. If the number of mutation positions is 4, the current score set may be represented in a matrix form. In the matrix corresponding to the current score set, (row u, column w) represents a current score corresponding to a u^{th} amino acid at a w^{th} mutation position, 1≤u≤m, and 1≤w≤k, where m is the number of amino acid types, for example, 20, and k represents the number of mutation positions, for example, 4. For example, if the amino acid at a first mutation position in the mutant protein is a first amino acid, the corresponding current score is a score in (row 1, column 1) in the matrix.

In the unsaturated mutagenesis scenario, the current score set is a current score vector, and a u^{th} element in the current score vector is a score of a u^{th} amino acid. If the mutant protein has two mutation positions and amino acids at the two mutation positions are a third amino acid and a tenth amino acid respectively, a score corresponding to the third amino acid is a score corresponding to a third position in the current score vector, and a score corresponding to the tenth amino acid is a score corresponding to a tenth position in the current score vector.

In some embodiments, in the k-site saturated mutagenesis scenario, the server may determine the reference object set through the following algorithm:
Input data of the algorithm includes: p, set Dₜᵣₐᵢₙ={(S₀, y₀)}, and matrix M,
where p refers to the number of occurrences of each amino acid at each mutation site, namely, the target frequency, which is, for example, 2. Dₜᵣₐᵢₙ refers to the first protein set, S₀ in (S₀, y₀) represents the wild-type protein, and y₀ represents the index experimental value of the wild-type protein. M refers to the current score matrix, and M∈ [symbol] ^{m×L}, where m is the number of amino acid types, for example 20, and AAINDEX (a) represents coordinates of amino acid a in matrix M, namely, the position of a score corresponding to amino acid a in M.

The step of initializing the current score matrix is: if a u^{th} amino acid occurs at a w^{th} mutation position of S₀, M_{uw}=p−1, otherwise M_{uw}=p. M_{uw} is an element in (row u, column w) in M, where 1≤w≤k.

Output data of the algorithm includes: an updated set Dₜᵣₐᵢₙ. Dₜᵣₐᵢₙ outputted by the algorithm is an initial reference object set.

The algorithm includes the following steps:

Step 1: while ∃M_{uw}>0 do. This step means that: step 2 to step 5 will be performed if matrix M has elements less than 0.

Step 2: Calculate a score of each mutant: Scoreᵢ=Σ_{(u,w)∈V}_{ᵢ}(M_{uw}).

Scoreᵢ is a current protein score of an i^{th} mutant protein Sᵢ in the first object set, and Vᵢ={(u,w)| AAINDEX (Sᵢⱼ)} represents coordinates (u, w) of a current score corresponding to each amino acid Sᵢⱼ at each mutation position in Sᵢ in matrix M.

Step 3: Select a mutant protein i*=argmaxScoreᵢ with the maximum score. This step is used for determining the mutant protein with the maximum current protein score.

i* represents that the mutant protein with the maximum current protein score is an i*^{th} mutant protein in the first object set.

Step 4: Update a set Dₜᵣₐᵢₙ←(Sᵢ*,yᵢ*). This step means that: the i*^{th} mutant protein in the first object set is added to the first protein set.

Sᵢ* in (Sᵢ*,yᵢ*) represents the i*^{th} mutant protein in the first object set, and yᵢ* represents the index experimental value of the i*^{th} mutant protein.

Step 5: Update the score matrix M, if the u^{th} amino acid occurs at the w^{th} mutation position of Sᵢ*, M_{uw}=M_{uw}−1, otherwise M_{uw}=M_{uw}.

Step 6: end while. Step 7 will be performed if M does not have elements greater than 0.

Step 7: Output Dₜᵣₐᵢₙ.

In some embodiments, in the unsaturated mutagenesis scenario, the server may obtain the reference object set by screening through the following algorithm:
Input data of the algorithm includes: p, set Dₜᵣₐᵢₙ={(S₀, y₀)}, and vector Q,
where p refers to the number of occurrences of each amino acid at each mutation site, namely, the target frequency, which is, for example, 2. Dₜᵣₐᵢₙ refers to the first protein set, S₀ in (S₀, y₀) represents the wild-type protein, and y₀ represents the index experimental value of the wild-type protein. Vector Q refers to the current score vector, and Q∈R^{m}, where m is the number of amino acid types, for example 20, and AAINDEX (a) represents coordinates of amino acid a in vector Q, namely, the position of a score corresponding to amino acid a in Q.

The step of initializing the current score vector is: if a u^{th} amino acid occurs at a mutation position of S₀, Qᵤ=p−1, otherwise Qᵤ=p. Qᵤ is a u^{th} element in vector Q.

Output data of the algorithm includes: an updated set Dₜᵣₐᵢₙ. Dₜᵣₐᵢₙ outputted by the algorithm is an initial reference object set.

Step 1: while ∃Qᵤ>0 do. This step means that: step 2 to step 5 will be performed if matrix Q has elements less than 0.

Step 2: Calculate a score of each mutant: Scoreᵢ=Σ_{k∈B}_{ᵢ}(Qᵤ).

Scoreᵢ is a current protein score of an i^{th} mutant protein Sᵢ in the first object set, and Bᵢ={u|AAINDEX (Sᵢⱼ)} represents coordinates u of a current score corresponding to each amino acid Sᵢⱼ at each mutation position in Sᵢ in matrix Q.

Step 3: Select a mutant i*=argmaxScoreᵢ with the maximum score. This step is used for determining the mutant protein with the maximum current protein score.

i* represents that the mutant protein with the maximum current protein score is an i*^{th} mutant protein in the first object set.

Step 4: Update a set Dₜᵣₐᵢₙ←(Sᵢ*, yᵢ*). This step means that: the i*^{th} mutant protein in the first object set is added to the first protein set.

Step 5: Update the score matrix Q. If a u^{th} amino acid occurs at a mutation position of Sᵢ*, Qᵤ=p−1, otherwise Qᵤ=p. Qᵤ is a u^{th} element in vector Q.

Step 6: end while. Step 7 will be performed if Q does not have elements greater than 0.

Step 7: Output Dₜᵣₐᵢₙ.

In this embodiment, since each amino acid corresponds to an amino acid, the score in the current score set is uniquely identified by the amino acid and the mutation position. For the amino acid at each mutation position, a current score corresponding to the amino acid at the mutation position is determined from the current score set according to the amino acid corresponding to the amino acid and the mutation position, so that the current score of each amino acid at each mutation position may be accurately and quickly determined.

According to the method for protein encoding (namely, the method for determining protein features) provided by this application, protein evolution may be assisted in combination with Bayesian optimization. The process of encoding proteins to obtain protein features may be referred to as the process of protein feature representation. The effective protein feature representation is very important for Bayesian optimization to find the best protein mutant. In order to better combine domain knowledge to construct an accurate and informative low-dimensional feature representation, a new low-dimensional encoding policy is proposed in this application to represent each amino acid at each site. Specifically, for two experimental scenarios in protein directed evolution: k-site saturated mutagenesis scenario and unsaturated mutagenesis scenario, two ways are made to calculate the amino acid representation of each site.

In some embodiments, in response to the object feature being a protein feature, the determining the object features of the objects in the reference object set based on the index experimental value of each object in the reference object set on the preset index includes: dividing, for each mutation position, the reference object set according to the type of amino acids at the mutation positions to obtain a first sub-object set corresponding to each amino acid; determining, for each amino acid at each mutation position, an amino acid feature of the amino acid at the mutation position based on an index experimental value of each object in the first sub-object set corresponding to the amino acid; and obtaining a protein feature of the object based on the amino acid feature of the amino acid at each mutation position in the object.

The first sub-object set is a sub-set of the reference object set. The first sub-object set includes at least part of the objects in the reference object set. The first sub-object set is uniquely determined by mutation positions and types of amino acids. For example, there are four mutation positions: a first mutation position, a second mutation position, a third mutation position, and a fourth mutation position, and there are 20 amino acids: ii amino acids, where 1≤ii≤20. Then 80 first sub-object sets are generated. At least one of mutation positions and amino acids corresponding to different first sub-object sets is different. For example, first sub-object set 1 is a first sub-object set corresponding to a first mutation position and a first amino acid, and first sub-object set 2 is a first sub-object set corresponding to a first mutation position and a second amino acid.

Specifically, the server may divide, for each mutation position, the reference object set according to the type of amino acids at the mutation positions to obtain a first sub-object set corresponding to each amino acid. For example, for a kk^{th} mutation position, the server may acquire an amino acid at the kk^{th} mutation position from each object in the reference object set to form an amino acid set corresponding to the kk^{th} mutation position. For example, if a parameter object set includes 40 objects, the amino acid set includes 40 amino acids, and the types of amino acids at the kk^{th} mutation position in different objects may be the same or different. After obtaining the amino acid set corresponding to the kk^{th} mutation position, the server may divide the amino acid set into a plurality of sub-sets according to the types of amino acids, divide the same amino acid into the same sub-set, and divide different amino acids into different sub-sets. Each sub-set only includes one amino acid. Each of the divided sub-sets is the first sub-object set corresponding to each amino acid at the kk^{th} mutation position. For example, if a j^{th} position in a protein is a mutation position, a first sub-object set corresponding to the mutation position may be represented as Vⱼ (a)={i|Sᵢⱼ=a}, where j represents the mutation position, i represents the number of objects in the reference object set, and Sᵢⱼ represents the amino acid at mutation position j in an i^{th} object Sᵢ in the reference object set. a represents any amino acid. For example, if there are 20 amino acids, a represents any one of the 20 amino acids. If a is the first amino acid (denoted as A1), the first sub-object set corresponding to amino acid A1 at mutation position j is Vⱼ (A1)={i|Sᵢⱼ=A1}.

In some embodiments, the server may determine, for each amino acid at each mutation position, an amino acid feature of the amino acid at the mutation position based on an index experimental value of each object in the first sub-object set corresponding to the amino acid. For example, if the first sub-object set corresponding to amino acid A1 at mutation position j is Vⱼ (A1)={i|Sᵢⱼ=A1}, an amino acid feature of amino acid A1 at mutation position j may be determined by using the index experimental value of the object corresponding to the number of each object in Vⱼ (A1)={i|Sᵢⱼ=A1} when calculating the amino acid feature of amino acid A1 at mutation position j.

In this embodiment, for each amino acid at each mutation position, the amino acid feature of the amino acid at the mutation position is determined based on the index experimental value of each object in the first sub-object set corresponding to the amino acid. Therefore, the amino acid features of the same amino acid at different mutation positions are related to the mutation positions. That is, the same amino acid has different feature representations at different positions. For example, the features of the same type of amino acids at different mutation positions may be different, thus improving the accuracy of amino acid encoding. The method for determining protein features provided by this embodiment may be applied to encoding a mutant protein generated in a k-site saturated mutagenesis scenario to obtain a protein feature of the mutant protein.

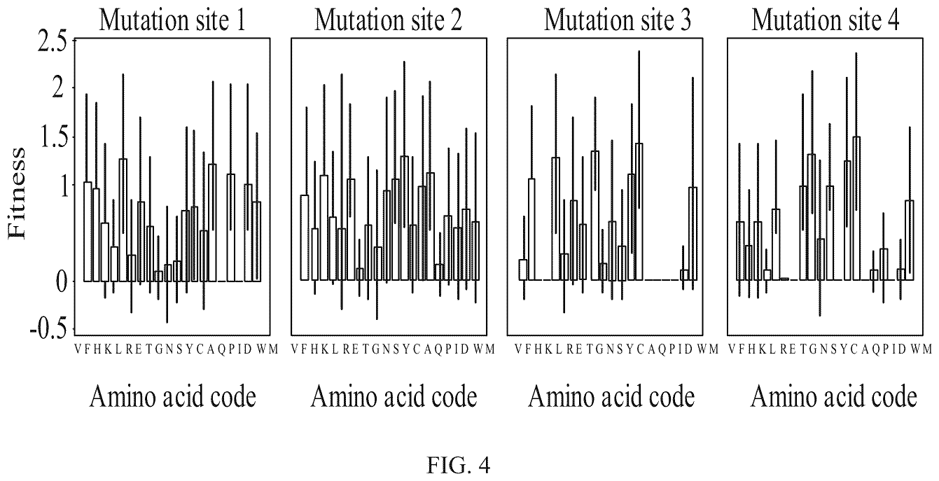

The object determining method provided by this application may be applied to Bayesian optimization, and Bayesian optimization is adopted to assist in protein directed evolution. The Bayesian optimization can effectively explore a combination space and find an optimal solution in a sample space by balancing exploration and utilization with as few experiments as possible in a small number of measurement samples. However, in the application of Bayesian optimization, the current encoding policy will inevitably encounter some problems. On the one hand, the high-dimensional encoding policy is challenging to Bayesian optimization, because an accurate and informative low-dimensional representation is required for successful global optimization search. On the other hand, classification tags (for example, one-hot encoding) may lead to the loss of knowledge about dead mutants from available experimental data for specific proteins. As can be seen in FIG. 4, each letter corresponding to the abscissa in FIG. 4 represents an amino acid. For example, V is an amino acid. FIG. 4 calculates a mean fitness of 20 amino acids (AA) at four mutation sites in 384 experimental samples (GB1 mutants) selected from a GB1 dataset. The mean fitness of each amino acid at each mutation site is obtained by calculating a mean of measurement values with affinity at the mutation site, and a corresponding standard deviation is shown as an error bar (namely, a vertical line in FIG. 4). As can be clearly seen from FIG. 4, the presence of some dead mutants at a particular mutation site will directly lead to low or zero fitness, regardless of the selection of amino acids at other sites. Therefore, the application of the existing protein encoding methods to assist in protein directed evolution in Bayesian optimization is usually ineffective.

However, according to the protein encoding method (namely, the method for determining protein features) provided by this application, the protein features obtained by encoding are accurate and informative low-dimensional features, so that the object determining method provided by this application is applied to Bayesian optimization, and the Bayesian optimization may be quickly used for assisting in protein directed evolution.

In some embodiments, the determining an amino acid feature of the amino acid at the mutation position based on an index experimental value of each object in the first sub-object set corresponding to the amino acid includes: statistically calculating the index experimental value of each object in the first sub-object set corresponding to the amino acid to obtain at least one index experimental statistical value; and determining the amino acid feature of the amino acid at the mutation position based on the at least one index experimental statistical value.

There may be one or more index experimental statistical values. More index experimental statistical values mean at least two index experimental statistical values. The statistical calculation includes, but is not limited to, calculating at least one of a mean, a minimum, or a maximum.

Specifically, the server may statistically calculate the index experimental value of each object in the first sub-object set corresponding to the amino acid to obtain at least one index experimental statistical value, and determine the amino acid feature of the amino acid at the mutation position based on the at least one index experimental statistical value.

In some embodiments, the statistically calculating the index experimental value of each object in the first sub-object set corresponding to the amino acid to obtain at least one index experimental statistical value includes: performing mean calculation on the index experimental value of each object in the first sub-object set corresponding to the amino acid to obtain a first index mean; and determining a maximum index experimental value to obtain a first index maximum from the index experimental value of each object in the first sub-object set corresponding to the amino acid, the at least one index experimental statistical value including at least one of the first index mean or the first index maximum. For example, if the first sub-object set corresponding to amino acid A1 at mutation position j is Vⱼ (A1)={i|Sᵢⱼ=A1}, index experimental values of objects corresponding to numbers in Vⱼ (A1)={i|Sᵢⱼ=A1} are acquired when calculating the amino acid feature of amino acid A1 at mutation position j, a mean of the acquired index experimental values is calculated to obtain a first index mean, and a maximum is determined from the index experimental values to obtain a first index maximum. The first index mean is an index experimental statistical value, and the maximum is also an index experimental statistical value. Based on at least one of the first index mean or the first index maximum, the amino acid feature of amino acid A1 at mutation position j is determined. In this embodiment, since the first index mean and the first index maximum are obtained statistically, the characteristics of amino acids may be reflected, so that the first index mean or the first index maximum are taken as the index experimental statistical value, thus improving the accuracy of the index experimental statistical value.

In some embodiments, the determining the amino acid feature of the amino acid at the mutation position based on the at least one index experimental statistical value includes: combining the first index mean and the first index maximum into the amino acid feature of the amino acid at the mutation position. For example, the first index mean and the first index maximum may be used as feature values to form an amino acid feature. That is, the amino acid feature includes the first index mean and the first index maximum. For example, the first index mean may be represented as Formula (1), and the first index maximum may be represented as Formula (2). yᵢ in Formula (1) and Formula (2) represents the index experimental value of an i^{th} object on the preset index in the reference object set. a in Formula (1) and Formula (2) represents the amino acid. In this embodiment, the first index mean and the first index maximum are combined into the amino acid feature of the amino acid at the mutation position, so that the amino acid feature is related to the index experimental value, thereby improving the accuracy of a target object screened based on the amino acid feature.

$${E}_{j}^{mean} ( a ) = \frac{1}{\vert {V}_{j} ( a ) \vert} {\sum }_{i ∈ {V}_{j} ( a )} {y}_{1} , \qquad ( 1 )$$

$${E}_{j}^{max} ( a ) = {max}_{i ∈ {V}_{j} ( a )} {y}_{i} \qquad ( 2 )$$

For example, the mutant protein in the reference object set is a mutant protein generated in the k-site saturated mutagenesis scenario. The amino acids are encoded by calculating a mean or a maximum of measurement values of the affinity of the mutant protein at which each amino acid at each mutation site is located. The features of the corresponding mutant proteins are represented by feature vectors encoded by the amino acids. This method allows the same amino acid to have different feature representations at different positions, and creates a smoother local variable for regression.

In this embodiment, the index experimental value of each mutant protein in the first sub-object set corresponding to the amino acid is statistically calculated, the amino acid feature of the amino acid at the mutation position is determined based on the statistical index experimental statistical value, and the accuracy of the amino acid feature obtained by encoding is improved through statistical data. The method for determining protein features provided by this embodiment may be applied to encoding a mutant protein generated in a k-site saturated mutagenesis scenario to obtain a protein feature of the mutant protein.

In some embodiments, in response to the object feature being a protein feature, the determining the object features of the objects in the reference object set based on the index experimental value of each object in the reference object set on the preset index includes: determining, for each amino acid, an object in which an amino acid at a mutation position includes the amino acid from the reference object set to obtain a second sub-object set corresponding to the amino acid; determining, for each amino acid, an amino acid feature of the amino acid based on index experimental values of objects in the second sub-object set corresponding to the amino acid; and obtaining a protein feature of the object based on the amino acid feature of the amino acid at each mutation position in the object.

The second sub-object set is a sub-set of the reference object set. The second sub-object set includes at least part of the objects in the reference object set. Each second sub-object set corresponds to an amino acid respectively. Different second sub-object sets correspond to different amino acids.

Specifically, the objects in the reference object set are proteins, and the reference object set may include mutant proteins and wild-type proteins. The server may determine, for each amino acid, an object in which an amino acid at a mutation position includes the amino acid from the reference object set to form a second sub-object set corresponding to the amino acid. For example, for a first amino acid A1, amino acids at each mutation position of each object are determined to form an amino acid set corresponding to the object. After the amino acid sets corresponding to the objects in the reference object set are obtained, an amino acid set including the first amino acid A1 is determined from the amino acid sets corresponding to the objects, and the objects corresponding to the amino acid set including A1 are combined into a second sub-object set corresponding to the amino acid A1.

In some embodiments, for each object in the reference object set and for each amino acid, the server may determine a mutation position corresponding to each amino acid from the object. When the amino acid at mutation position 1 is A1, the mutation position corresponding to amino acid A1 is mutation position 1. Each amino acid may correspond to 0, 1 or more mutation positions, and more mutation positions mean at least two mutation positions. For example, for an i^{th} object Sᵢⱼ in the reference object set, a set Nᵢ(a) of mutation positions corresponding to each amino acid may be represented as Formula (3), where j is the mutation position. For each amino acid, a second sub-object set corresponding to each amino acid may be determined based on the set of mutation positions corresponding to each amino acid in the amino acids. For example, the second sub-object set V(a) may be represented as Formula (4). In Formula (4), for amino acid a, if the number (namely, |Nᵢ (a)|) of mutation positions corresponding to amino acid a in an i^{th} object is not 0, the i^{th} object is taken as an object in the second sub-object set corresponding to amino acid a. |Nᵢ(a)| represents the number of elements included in set Nᵢ(a).

$${N}_{i} ( a ) = \vert \{ j \vert {S}_{ij} = a \} \vert \qquad ( 3 )$$

$$V ( a ) = \{ i \vert \vert {N}_{i} ( a ) \vert \neq 0 \} \qquad ( 4 )$$

In some embodiments, the server may statistically calculate, for each amino acid, index experimental values of objects in the second sub-object set corresponding to the amino acid to obtain an amino acid feature of the amino acid. The statistical calculation includes, but is not limited to, calculating at least one of a mean, a maximum, or a minimum. For example, the server may calculate a mean of the index experimental values of the objects in the second sub-object set to obtain a second index mean, acquire a maximum index experimental value in the index experimental values of the objects in the second sub-object set to obtain a second index maximum, and obtain the amino acid feature of the amino acid based on at least one of the second index mean or the second index maximum. For example, the second index mean and the second index maximum may be used as feature values to form the amino acid feature. That is, the amino acid feature includes the second index mean and the second index maximum. For example, the second index mean may be represented as Formula (5), and the second index maximum may be represented as Formula (6). yᵢ in Formula (5) and Formula (6) represents the index experimental value of an i^{th} object on the preset index in the reference object set.

$${E}^{mean} ( a ) = \frac{1}{\vert V \vert} {\sum }_{i ∈ V} {y}_{1} , \qquad ( 5 )$$

$${E}^{max} ( a ) = {max}_{i ∈ V} {y}_{i} \qquad ( 6 )$$

In some embodiments, the server may obtain, based on each mutation position in a mutant protein and the amino acid feature of the amino acid at each mutation position, a protein feature of the mutant protein by encoding. For example, for a mutant protein generated in the unsaturated mutagenesis scenario, if each mutant protein includes two mutation positions, a vector composed of the two mutation positions and the amino acid features of the amino acids corresponding to the two mutation positions is determined as a protein feature of the mutant protein. Therefore, for a mutant protein generated in the unsaturated mutagenesis scenario, an amino acid in the mutant protein may be encoded by calculating a mean or maximum of fitness measurement values (index experimental values) of proteins containing the amino acid at any position. An expression vector of the mutant protein is encoded from mutant positions and corresponding mutant amino acids. The encoding mode is more in line with the biological significance of protein evolution and greatly reduces the dimension of feature.

In this embodiment, since the amino acid at the mutation position of each mutant protein in the second sub-object set corresponding to an amino acid includes the amino acid, for each amino acid, an amino acid feature of the amino acid is obtained based on the index experimental values of the mutant proteins in the second sub-object set corresponding to the amino acid, so that the amino acid is encoded based on the index experimental value of the protein including the amino acid, and the accuracy of amino acid encoding is improved. The method for determining protein features provided by this embodiment may be applied to encoding a mutant protein generated in an unsaturated mutagenesis scenario to obtain a protein feature of the mutant protein.

In some embodiments, the determining a target object meeting index requirements of the preset index from the second object set based on the mapping relationship includes: determining a statistical index value of each object in the second object set on a target statistical index based on the mapping relationship, and determining a selected object from the second object set based on the statistical index value; adding the selected object to the reference object set when iteration stop conditions are not satisfied; redetermining the object features of the objects in the reference object set based on the index experimental value of each object in the reference object set on the preset index until the iteration stop conditions are satisfied; and determining the selected object obtained when the iteration stop conditions are satisfied as the target object meeting the index requirements of the preset index.

The mapping relationship between the preset index and the object features is a first mapping relationship. The iteration stop conditions include that the number of iterations (namely, the number of cycles) reaches a number threshold and/or the index experimental value of the selected object reaches a second index threshold. The selected object may be constantly changing, and selected objects determined in different cycles are different.

Specifically, the server may perform statistical calculation based on the first mapping relationship to obtain a second mapping relationship between a target statistical index and the object features, and determine a statistical index value of each object in the second object set on the target statistical index based on the second mapping relationship. The statistical index value refers to a value of an object on the target statistical index. For example, the second mapping relationship is represented by a curve y2=f2 (x). In order to determine the index statistical value of the object on the target statistical index, a value of y2 may be calculated when x in the curve y2=f2 (x) is the object feature of the object, and the value of y2 may be determined as the index statistical value of the object on the target statistical index.

In some embodiments, there may be one or more selected objects. The object corresponding to the largest statistical index value may be determined as the selected object. Alternatively, the object with the statistical index value greater than a third index threshold may be determined as the selected object, and the third index threshold may be set as required. The server may obtain an object meeting the index requirements of the preset index based on the selected object. For example, the server may determine the selected object as the object meeting the index requirements of the preset index.

In some embodiments, the server may add the selected object to the reference object set when iteration stop conditions are not satisfied, redetermine the object features of the objects in the reference object set based on the index experimental value of each object in the reference object set on the preset index until the iteration stop conditions are satisfied, and determine the selected object obtained when the iteration stop conditions are satisfied as the target object meeting the index requirements of the preset index. For example, if the iteration stop conditions are that the number of iterations (namely, the number of cycles) reaches a number threshold, the selected object is determined as the target object when the number of iterations (namely, the number of cycles) reaches the number threshold.

In this embodiment, when the iteration stop conditions are not satisfied, a new selected object is determined again, so as to gradually find the target object meeting the index requirements of the preset index. Since the selected object is added to the reference object set, the number of objects in the reference object set is increased. Therefore, in the process of determining a new selected object every time, the step of determining the object features of the objects in the reference object set is performed, thereby gradually improving the accuracy of the object features obtained by encoding, and improving the accuracy of the finally selected target object.

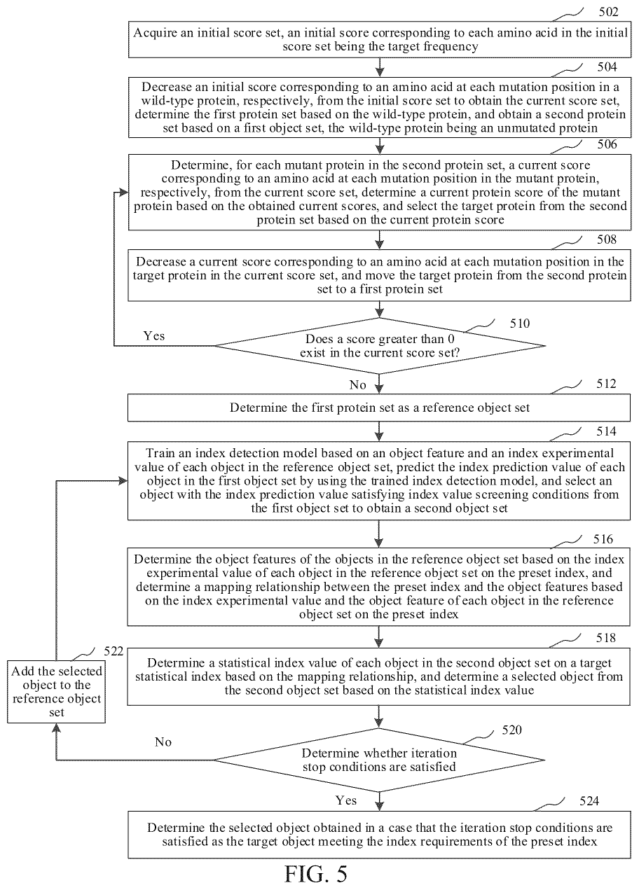

In some embodiments, as shown in FIG. 5, an object determining method is provided. An object in the method is a mutant protein. The method may be performed by a terminal or a server, and may also be performed jointly by the terminal and the server. The method, illustrated by being applied to a server, includes the following steps:

Step 502: Acquire an initial score set, an initial score corresponding to each amino acid in the initial score set being the target frequency. Step 504: Decrease an initial score corresponding to an amino acid at each mutation position in a wild-type protein, respectively, from the initial score set to obtain the current score set, and determine the first protein set based on the wild-type protein, the wild-type protein being an unmutated protein. A second protein set is obtained based on a first object set.

Step 506: Determine, for each mutant protein in the second protein set, a current score corresponding to an amino acid at each mutation position in the mutant protein, respectively, from the current score set, determine a current protein score of the mutant protein based on the obtained current scores, and select the target protein from the second protein set based on the current protein score.

Step 508: Decrease a current score corresponding to an amino acid at each mutation position in the target protein in the current score set, and move the target protein from the second protein set to a first protein set.

Step 510: Determine whether a score greater than 0 exists in the current score set, return to perform step 506 if the score exists, otherwise, perform step 512.

Step 512: Determine the first protein set as a reference object set.

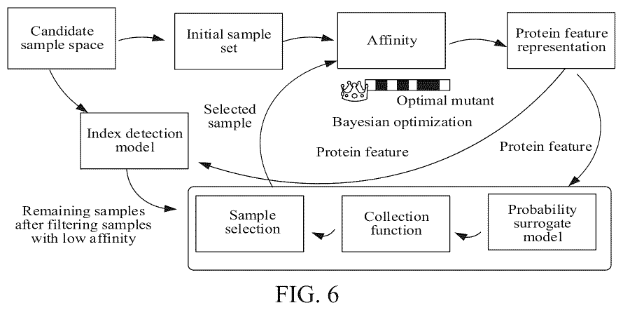

FIG. 6 shows a schematic diagram of an object determining method for determining mutant proteins with high affinity. A candidate sample space includes wild-type proteins and a plurality of mutant proteins. The reference object set in Step 508 is an initial reference object set, and the reference object set will change afterwards. The initial reference object set is, for example, an initial sample set in FIG. 6. According to the method for determining the initial reference object set provided by this application, an initial sample set is screened from the candidate sample space, and samples in the initial sample set are any one of wild-type proteins or mutant proteins. Step 514: Train an index detection model based on an object feature and an index experimental value of each object in the reference object set, predict the index prediction value of each object in the first object set by using the trained index detection model, and select an object with the index prediction value satisfying index value screening conditions from the first object set to obtain a second object set.

As shown in FIG. 6, after the initial sample set is obtained, an “affinity” stage is used for acquiring affinity of the samples in the initial sample set (measured experimentally). After obtaining the affinity, the proteins in the initial sample set are encoded in a “protein feature representation” stage to obtain the protein features of the proteins in the initial sample set. The index detection model is trained by using the protein affinity (measured experimentally) and the protein features. After training, the protein features corresponding to the proteins in the candidate sample space are inputted into the index detection model, an affinity prediction value of the protein is predicted, and the proteins with the affinity prediction values greater than an affinity threshold are selected from the candidate sample space to form the second object set.

A search space pre-screening policy is provided by screening the initial sample set based on the candidate sample space (namely, screening the second object set based on the first object set). Many mutants in the candidate sample space have low affinity values. By adopting the search space pre-screening policy of samples, the samples with low affinity values are eliminated in advance, thereby reducing a sample space to be searched in Bayesian optimization, and improving the calculation efficiency. For example, mutants with low affinity may be eliminated from the sample search space by using XGBOD. Specifically, in each iteration process of Bayesian optimization, the samples in the candidate sample space may be pre-screened first. By setting a threshold, the samples lower than the threshold are determined as low fitness (positive class), and the samples higher than the threshold are determined as high fitness (negative class). Samples with existing experimental values (that is, proteins with affinity has been determined experimentally) are used as training sets to train XGBOD, samples in the candidate sample space are screened, and potential sample points with low affinity are filtered out in advance, so as to reduce the sample size in a sampling space and improve the model efficiency.

Each process of determining a selected object from the second object set may be regarded as a Bayesian optimization. For example, in FIG. 6, the process of screening samples from the remaining samples after filtering out the samples with low affinity by using a collection function is a Bayesian optimization.

Step 516: Determine the object features of the objects in the reference object set based on the index experimental value of each object in the reference object set on the preset index, and determine a mapping relationship between the preset index and the object features based on the index experimental value and the object feature of each object in the reference object set on the preset index.

The mapping relationship between the preset index and the object features is, for example, a probability surrogate model in FIG. 6. The probability surrogate model may be any one of a Gaussian process regression model based on Gaussian distribution, a Gaussian process regression model based on student-t distribution, or the like.

Step 518: Determine a statistical index value of each object in the second object set on a target statistical index based on the mapping relationship, and determine a selected object from the second object set based on the statistical index value.

As shown in FIG. 6, the target statistical index is, for example, a collection function in FIG. 6. The collection function is determined by using the mapping relationship between the preset index and the object features, and samples are selected from the remaining samples based on the collection function.

Step 520: Determine whether iteration stop conditions are satisfied, if yes, perform step 522, otherwise, perform step 524.

Step 522: Add the selected object to the reference object set, and return to perform step 514.

Step 524: Determine the selected object obtained when the iteration stop conditions are satisfied as the target object meeting the index requirements of the preset index.

For example, the server may implement the process of determining the target object from the second object set based on the reference object set by using the following algorithm, which is Bayesian optimization with prescreened search space via outlier detection (ODBO).

Input data of the algorithm includes: an initial sample set D; and the number of experiments T (namely, the number of cycles T).

The initial sample set Dₜ is the initial reference object set, namely, the reference object set adopted in the first iteration.

Output data of the algorithm includes: an optimal value (s*, y*) in the sample space, where s* represents an optimal mutant and y* represents the affinity of s* (measured experimentally).

The algorithm includes the following process:

```
  t + 1; //assign 1 to t, where t represents the number of current iterations;
  while t ≤ T do; //execute downward when t ≤ T, where T is the number of
experiments (namely, the number of cycles);
    if Robust GP then; //execute downward if Robust GP is used as the
probability surrogate model;
        train the Gaussian process regression model based on student-t
distribution by using Dₜ; //Dₜ is equal to D₁ when t is 1, indicating that the initial sample set is
the initial reference object set (the reference object set adopted in the first iteration);
        Dₜᵢₙ = {(Sᵢ, yᵢ) | |fₜ(Sᵢ) − yᵢ| ≤ α}, filter out outliers and retain inliers
according to a rejection threshold α; //Dₜᵢₙ represents a set of samples remaining after filtering
out outliers (samples) from Dₜ;
        train the Gaussian process regression model based on Gaussian
distribution by using Dₜᵢₙ;
    else if GP then; //execute downward if Robust GP is used as the probability
surrogate model;
        train the Gaussian process regression model based on Gaussian
distribution by using Dₜ;
    if Naive BO then; //execute downward if Naive Bayesian optimization is
adopted, Naive BO refers to Naive Bayesian optimization;
      $maximize a collection function {S}_{t + 1} = {arg max}_{S ∈ D \_ search} α ( S \vert {D}_{t} ) according to$
a current posterior probability//D_search refers to the second object set, namely, a set of
samples remaining after filtering out the samples with low affinity from the candidate sample
space; Sₜ₊₁ is a sample screened from the second object set (namely, the selected object);
        evaluate a data point Sₜ₊₁ experimentally and update the experimental
results to an observed sample set Dₜ₊₁ ← (Sₜ₊₁, yₜ₊₁)//yₜ₊₁ is the affinity of Sₜ₊₁ determined
(measured experimentally);
    else if TuRBO then; //trust region Bayesian optimization (TuRBO);
        set a confidence threshold interval Ω according to a current confidence
threshold TR;
        randomly sample a plurality of points within a trust region Ω, and
$maximize a collection function {S}_{t + 1} = {arg max}_{S ∈ Ω} α ( S \vert {D}_{t} ) according to a current posterior$
probability;
        evaluate a data point Sₜ₊₁ experimentally and update the experimental
results to an observed sample set Dₜ₊₁ ← (Sₜ₊₁, yₜ₊₁);
    end if; //end this cycle;
    update the probability surrogate model;
      t ← t + 1//increase t by 1;
  end while;
  output (s*, y*).
```

TuRBO is a global optimization method. By constructing a series of local GPs surrogate models, excessive exploration of highly uncertain regions in the search space can be avoided from a global perspective, and the second-order convergence of trust methods can be fully utilized for efficient solution locally.

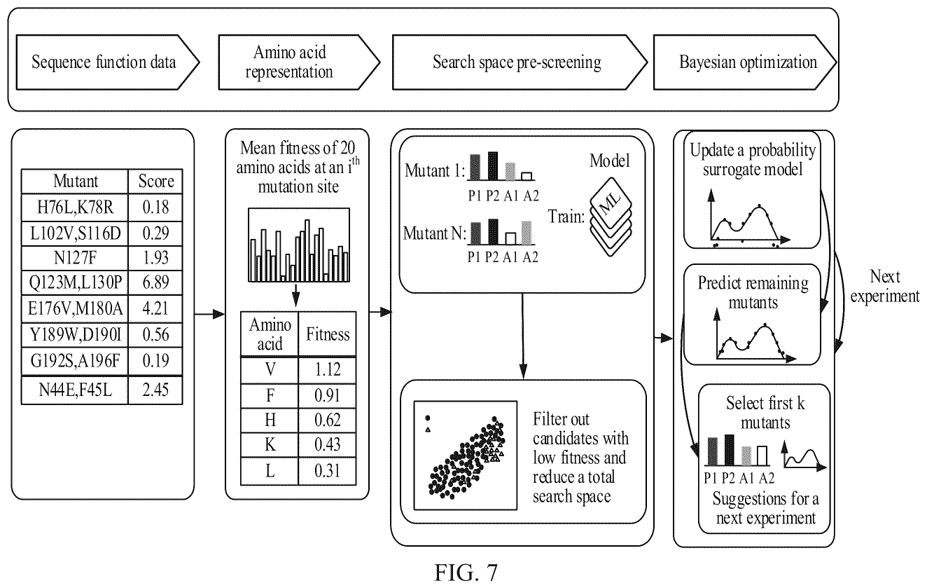

The basic process of the object determining method provided by this application is illustrated. As shown in FIG. 7, the basic process mainly includes four steps: 1) Acquire initial experimental data. 2) Characterize the data. 3) Pre-screening a search space. 4) Train, for the screened search space, a probability surrogate model by a Bayesian optimization algorithm through the initial experimental data. After training the surrogate model, a next round of experimental samples are selected in the search space by optimizing a collection function. The proposed experimental design is verified, the experimental results are added to a training set, and the surrogate model is updated posteriorly. This process is repeated until the design is maximized, resources are exhausted, or space is explored to conditions where improvement is unlikely to be found. FIG. 7(a) shows the initial experimental data obtained, namely, the initial reference object set screened from the first object set. Eight mutants are shown in (a), and each mutant has a score, which represents fitness. For example, mutant “H76L, K78R” represents a mutant, and the score of “H76L, K78R” is 0.18. FIG. 7(b) is the characterization of data (namely, the process of encoding to determine amino acid features). The bar graph in FIG. 7(b) shows the mean fitness of 20 amino acids at an i^{th} mutation site, and the table in FIG. 7(b) shows the mean fitness of five amino acids at the i^{th} mutation site. For example, the mean fitness of amino acid “V” is 1.12. FIG. 7(c) shows a search space pre-screening process, in which the features of mutants in the initial experimental data are determined. In FIG. 7(c), “P1 P2 A1 A2” is the features of mutants, where P is the abbreviation of position, A is the abbreviation of Amino Acid, P1 and P2 represent the positions of amino acids in mutants respectively, and A1 and A2 represent the features of amino acids respectively. The search space is pre-screened by using the determined features of the mutants. In FIG. 7(c), solid circles represent outliers (namely, mutants with low fitness), and solid triangles represent inliers (namely, mutants with high fitness). FIG. 7(d) shows the process of the Bayesian optimization algorithm. Mutants with high fitness may be determined through the four processes in FIG. 7.

In view of computing method assisted experimental design, this application provides an efficient and experimental design-oriented framework. Through the search space pre-screening policy, the samples in the candidate sample space are pre-screened in advance (that is, the second object set is screened from the first object set). Combined with the Bayesian optimization algorithm, exploration and utilization are balanced, the sample space is effectively explored, and the optimal experimental design scheme is found in as few steps as possible. In this application, aiming at a practical application scenario of protein directed evolution, an amino acid encoding policy based on mean fitness is designed to accurately and effectively represent features (namely, the method for obtaining amino acid features). In order to better assist experimenters in experimental design, an initial sample selection policy is provided to assist experimenters in selecting initial experimental samples (namely, the method for determining the reference object set), so as to ensure the maximum coverage of amino acid encoding information covered in an initial sample size and the minimum number of initial experiments. Through the Bayesian optimization algorithm for pre-screening the search space, the experimental cost and the time cost are reduced. In this application, an efficient and experimental design-oriented framework is implemented, which is referred to as Bayesian optimization with prescreened search space via outlier detection (ODBO). This method assists in experiment design by search space screening and Bayesian optimization, and helps experimenters reduce the experimental cost and the time cost. Aiming at a practical application scenario of protein directed evolution, an amino acid encoding policy based on mean fitness is provided to accurately and effectively represent features. In order to better assist experimenters in experimental design, this scheme also provides an initial sample selection policy to assist experimenters in selecting initial experimental samples, so that the maximum coverage of amino acid encoding information covered in an initial sample size is ensured, the least experiments are required, and the experimental cost is reduced.

The present disclosure may be used for solving the experimental design of computing method assisted protein directed evolution. The proposed Bayesian optimization combined with search space pre-screening may also be applied to automatic experimental design in other fields, such as new material development and battery fast charging protocols.

In material science, it is expensive and time-consuming to produce materials with certain performance. With the increase of each new component or material parameter, the space of candidate experiments increases exponentially. For example, if about 10 experiments are required within the parameter range to research the influence of a new parameter (for example, introducing doping), 10\ possible experiments will be required for N parameters. With the emergence of each new parameter, the number of candidate experiments quickly exceeds the feasibility of exhaustive exploration. The diversity and complexity of material composition-structure-property (CSP) relationships include material-processing parameters and atomic disorder, thus making the research more confusing. Coupled with the scarcity of the best materials, these challenges threaten innovation and industrial progress. An auxiliary material discovery method based on Bayesian optimization may guide laboratory experimenters to design experiments, thereby balancing the experiments of exploring unknown functions by experiments and identifying extremum by prior knowledge, and can increase the speed of material discovery and spend fewer resources in the experiments of material exploration.

A lithium-ion battery is one of the most commonly used energy storage apparatuses for electric vehicles. With the development of battery chemistry technology, an important problem is how to determine a charging protocol effectively, so as to best balance the demand of fast charging and maximize the service life of the battery. However, it is not easy to determine a suitable charging protocol. On the one hand, the cycle life of a battery is estimated to take from several months to several years. On the other hand, the huge parameter adjustment space and the diversity of samples make the experiment more difficult. How to further reduce the parameter range and shorten the experimental time is very important for the development of lithium-ion batteries. The computing assisted experimental design method may be used for reducing the cost of experimental optimization, providing information for subsequent experimental decision-making by using the feedback of completed experiments, and balancing a relationship between experimental results and requirements. That is, an experimental parameter space with high uncertainty is tested and explored, and promising parameters are predicted according to the completed experimental results. Finally, the number and time of experiments can be reduced, the cost can be reduced, and an effective charging protocol can be found.

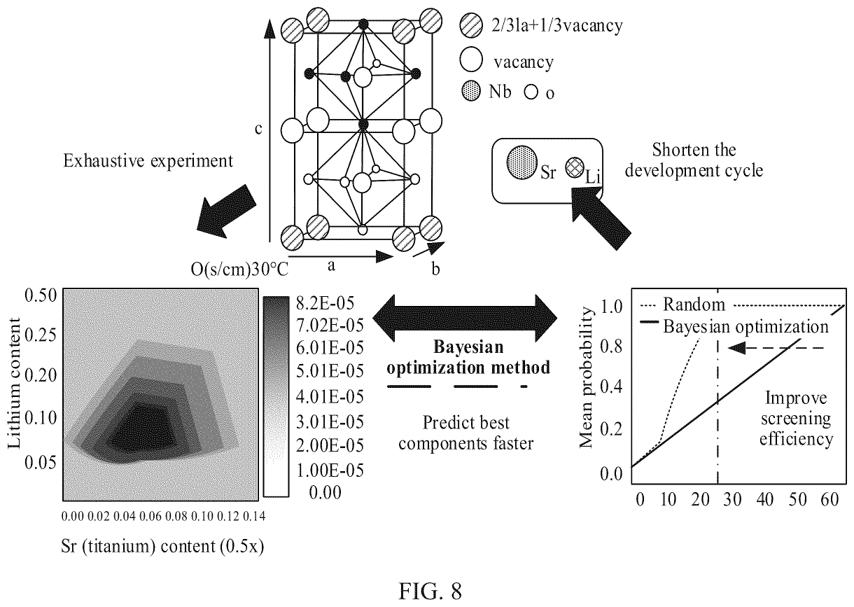

This application also provides an application scenario. The application scenario is a new material development scenario, and the application scenario applies the object determining method. Specifically, the application of the object determining method to the application scenario is as follows. The server may acquire index prediction values of materials in a first material set on a preset index respectively, select a material with the index prediction value satisfying index value screening conditions from the first material set to obtain a second material set, determine, based on index experimental values and material features of the plurality of materials in the first material set on the preset index, a mapping relationship between the preset index and the material features, and determine a target material meeting index requirements of the preset index from the second material set based on the mapping relationship. Therefore, the materials with specified performance are quickly determined. At least one component of each material in the first material set is different or the content of at least one component is different. FIG. 8 shows a closed-loop optimization of perovskite electrolyte based on machine learning. An effective experimental search for finding fast lithium-ion conductors from perovskite solid electrolyte is realized by using Bayesian optimization.

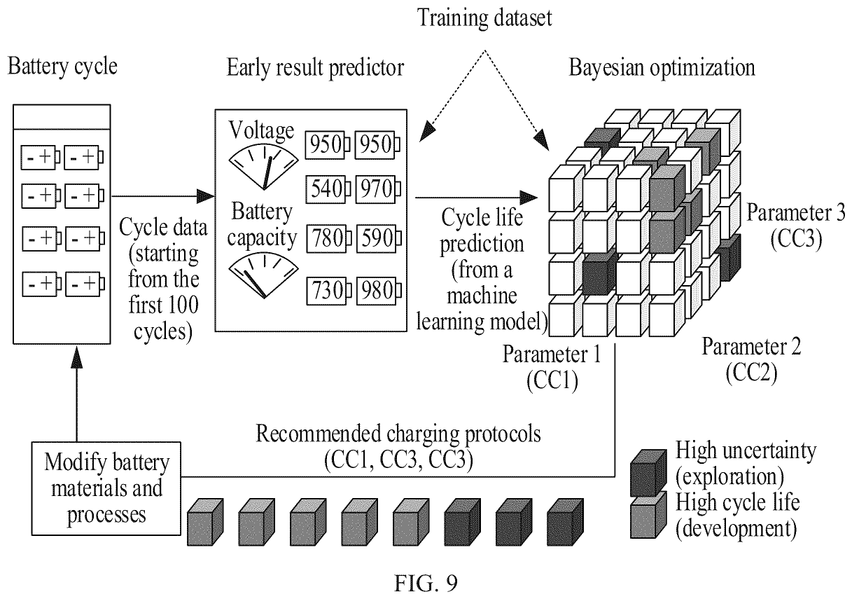

This application also provides an application scenario. The application scenario is a battery fast charging protocol scenario, and the application scenario applies the object determining method. Specifically, the application of the object determining method to the application scenario is as follows. The server may acquire index prediction values of battery charging protocols in a first battery charging protocol set on a preset index respectively, select a battery charging protocol with the index prediction value satisfying index value screening conditions from the first battery charging protocol set to obtain a second battery charging protocol set, determine, based on index experimental values and battery charging protocol features of the plurality of battery charging protocols in the first battery charging protocol set on the preset index, a mapping relationship between the preset index and the battery charging protocol features, and determine a target battery charging protocol meeting index requirements of the preset index from the second battery charging protocol set based on the mapping relationship. Therefore, the battery charging protocols with specified performance are quickly determined. At least one parameter of each battery charging protocol in the first battery charging protocol set is different or the value of at least one parameter is different. FIG. 9 shows a closed-loop optimization of a battery fast charging protocol based on machine learning. Through machine learning, the parameter space is effectively optimized, current and voltage configuration parameters of the fast charging protocol are specified, and the battery life is maximized.

The object determining method provided by this application may be deployed on a server equipped with Linux operating system or Windows operating system and CPU/GPU computing resources based on Python language and Botorch library.

In order to verify the effectiveness of the object determining method provided by this application in assisting protein directed evolution, tests were carried out on four protein directed evolution datasets: 1) GB1 dataset (with 55 mutation parts); 2) GB1 dataset (with 4 mutation parts); 3) BRCA1 dataset; and 4) green fluorescent protein dataset.

GB1 refers to a B1 structural domain of protein G. Protein G is an immunoglobulin binding protein, which is expressed in streptococci of group C and group G. The B1 structural domain (GB1) of protein G interacts with a Fc structural domain of immunoglobulin. The generated GB1 datasets were experimented respectively. Saturation mutagenesis was performed on four carefully selected residue sites 39, 40, 41, and 51 in GB1. There are experimentally measured fitness values in 149,361 mutants. The fitness standard is binding affinity with IgG-Fc. One or two amino acids were mutated in a random region of 55 codons of GB1 protein, and a total of 536,944 mutant data were collected.

BRCA1 is a multi-domain protein belonging to the tumor suppressor gene family, and mutations most often occur in three domains: N-terminal RING domain, exons 11-13 and BRCT domain. A BRCA1 RING structural domain is responsible for the E3 ubiquitin ligase activity of BRCA1 and mediates the interaction between BRCA1 and other proteins. The functional effects of single or multiple point mutations of BRCA1 residues on E3 ubiquitin ligase activity were researched. The dataset contains 98,300 mutants with E3 score.

Green fluorescent protein (GFP), also known as green fluorescent protein, was first found in a jellyfish with the scientific name Aequorea victoria (avGFP), which will show green fluorescence when exposed to light. The local fitness landscape of avGFP was analyzed by estimating fluorescence levels of genotypes obtained by random mutagenesis of avGFP sequences. The dataset includes 54,025 different protein sequences. The details of the four datasets used are shown in Table 1.

TABLE 1
Details of protein directed evolution dataset

| Protein | Target | Measure value | Length | Quantity | Maximum | Mean | Variance |
|---|---|---|---|---|---|---|---|
| GB1 | IgG-Fc | Fitness | 4 | 149361 | 8.76 | 0.08 | 0.4 |
| GB1 | IgG-Fc | Enrichment | 55 | 536944 | 2.53 | −2.42 | 2.28 |
| | | score | | | | | |
| Ube4b | E3 ubiquitin | log2 (E3 score) | 102 | 98300 | 9 | −0.87 | 1.34 |
| | ligase | | | | | | |
| avGFP | — | Brightness | 233 | 54025 | 4.12 | 2.63 | 1.06 |

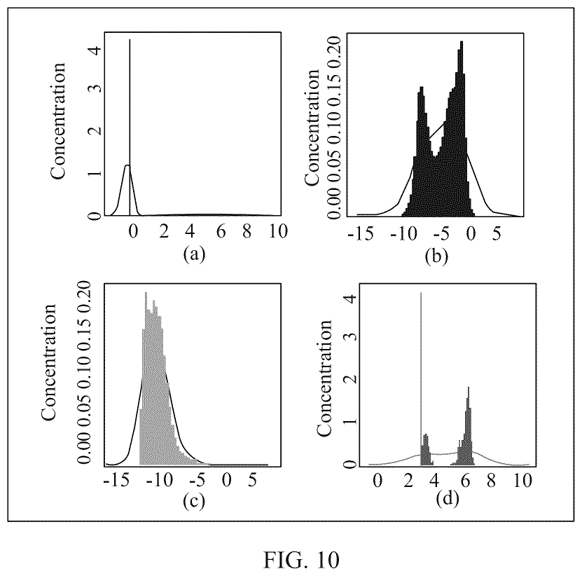

FIG. 10 shows a fitness distribution of different datasets. In FIG. 10, the Abscissa is the measure value, and the ordinate is Density (concentration or density). FIG. 10(a) is the fitness distribution of dataset GB1(4). FIG. 10(b) is the fitness distribution of dataset GB1(55). FIG. 10(c) is the fitness distribution of dataset BRCA1. FIG. 10(d) is the fitness distribution of dataset avGFP.

An initial sample set is generated by using an initial sample selection policy. For the GB1(4) dataset in a saturated mutation scenario, each amino acid occurs at each position at least twice, and 40 initial training samples are obtained. For the GB1(55), Ube4b, and avGFP datasets in an unsaturated mutation scenario, each amino acid occurs at all positions at least once, and 136, 217 and 142 initial training samples are obtained respectively. For an ODBO algorithm, a filtering threshold of search space pre-screening is set to 0.05. For each method, 10 different random seeds were used for each experiment. Each method selects one sample from the sample space each time in the GB1(55), Ube4b, and avGFP datasets and runs 50 iterations. For the GB1(55) dataset, one sample is selected from the sample space each time and 100 iterations are run. Expected improvement (EI) is used as the collection function. Ube4b and BRCA1 refer to the same protein.

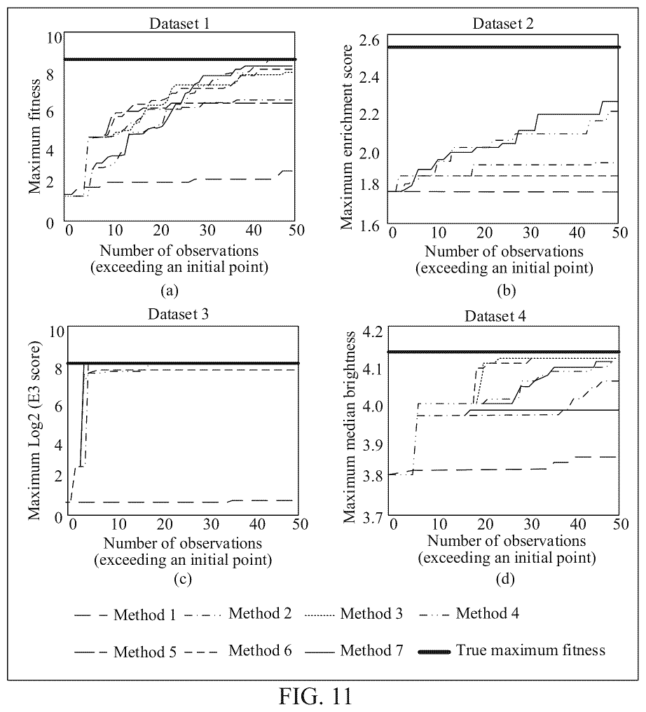

FIG. 11 summarizes the performance of different methods on four protein directed evolution datasets. Dataset 1 refers to dataset GB1(4). Dataset 2 refers to dataset GB1(55). Dataset 3 refers to dataset Ube4. Dataset 4 refers to avGFP. Method 1 refers to random. Method 2 refers to TuRBO combined with GP. Method 3 refers to ODBO combined with TURBO and GP. Method 4 refers to ODBO combined with TuRBO and RobustGP. Method 5 refers to Naïve BO combined with GP. Method 6 refers to ODBO combined with BO and GP. Method 7 refers to ODBO combined with BO and RobustGP. Each of the four figures in FIG. 11 includes a straight line, which refers to a true maximum fitness.

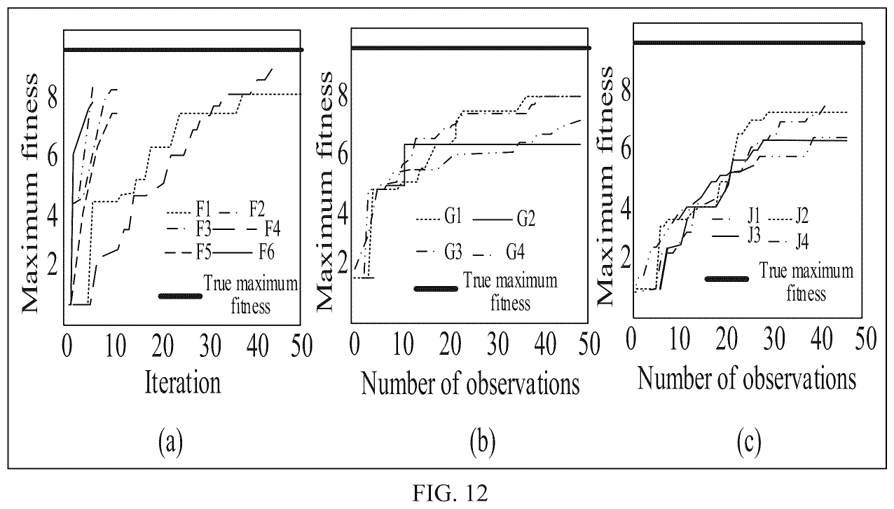

FIG. 12 summarizes the comparison of four protein directed evolution datasets by different methods. Each curve represents a mean obtained by the methods on 10 different random seeds. F1 represents ODBO combined with TuRBO and GP, and q=1. F2 represents ODBO combined with TuRBO and GP, and q=5. F3 represents ODBO combined with TuRBO and GP, and q=10. F4 represents ODBO combined with TURBO and RobustGP, and q=1. F5 represents ODBO combined with TuRBO and RobustGP, and q=5. F6 represents ODBO combined with TURBO and RobustGP, and q=10. G1 represents ODBO combined with TuRBO and GP, and the collection function is expected improvement. G2 represents ODBO combined with TURBO and GP, and the collection function is a confidence boundary policy. G3 represents ODBO combined with TuRBO and GP, and the collection function is Thompson sampling. q represents the number of samples selected for a next round of experiments in each iteration.

It can be found that ODBO achieves the best performance on all datasets. The search space pre-screening step may be used for collecting samples more effectively, which is helpful to find mutants with optimal properties faster. For example, in a saturated mutation scenario (namely, GB1 (4) dataset), ODBO combined with TURBO and RobustGP may find an optimal variable (fitness=8.76) through less than 50 evaluations in a large sample space (204=16000). However, Bayesian optimization algorithms (such as Naïve BO and TuRBO) usually converge to a bad local optimum without a pre-screening policy, thus reducing the mean performance. This shows the importance of search space pre-screening. In a case of unsaturated mutation, except Naïve BO combined with GP, almost all Bayesian optimization methods use the proposed low-dimensional protein encoding policy to find an optimal mutant. Although all methods can only find nearly optimal mutants in GB1(55) and avGFP datasets, the method proposed herein is also superior to other methods.

Table 2 shows a proportion of samples with the first 1%, 2% and 5% affinity values in a sample space screened by different calculation methods in GB1 (4) dataset in 50 rounds of recommended selections. It can be seen that using sample space pre-screening is more conducive to selecting a better sample from each round of sample selection for a next round of experimental tests.

TABLE 2

| Method | Top 1% | Top 2% | Top 5% |
|---|---|---|---|
| Random | 1.8 | 3.6 | 6.4 |
| Naive BO + GP | 14 | 20.6 | 31.2 |
| TuRBO + GP | 20.8 | 32.2 | 45 |
| ODBO, BO + GP | 29.6 | 41 | 62.2 |
| ODBO, TuRBO + GP | 31.6 | 44.6 | 67.2 |
| ODBO, BO + RobustGP | 35.6 | 50 | 65.8 |
| ODBO, TuRBO + RobustGP | 41.2 | 58.2 | 71.2 |

Furthermore, the performance of Bayesian optimization algorithms with different collection functions and probability surrogate models in protein directed evolution is also tested. FIG. 10 shows the performance of Bayesian optimization algorithms with different collection functions and probability surrogate models in a GB1 (4) dataset. The batch size of each iteration is different in FIG. 10(a). FIG. 10(b) shows the performance of using EI, UCB, PI, and TS as collection functions in the “ODBO, TURBO+GP” method. FIG. 10(c) shows the performance of using EI, UCB, PI, and TS as collection functions in the “ODBO, TuRBO+RobustGP” method.

Computing resources consumed by the methods running on the GB1 dataset are also calculated, as shown in Table 3. The traditional encoding mode (feature georgiev encoded by physical and chemical properties is shown here) has 76-dimensional features, and TURBO needs to consume a lot of computing resources and time. When amino acid encoding rules proposed herein are adopted, the feature dimension of amino acids may be reduced to 4, and the computing time and resource consumption can be greatly reduced. Furthermore, by adopting the search pre-screening policy, time and resources consumed by computing can be greatly reduced. Moreover, ODBO can find an optimal value in the sample space in the least experimental steps, which is helpful to reduce the experimental cost and the time cost.

TABLE 3

| | | Device 1 | | Device 2 | |
|---|---|---|---|---|---|
| | Feature | Number of | Elapsed | Number of | Elapsed |
| Method | dimension | CPUs | time | CPUs | time |
| ODBO | 4 | 1 | 56.31 | 1 | 62.53 |
| TuRBO | 4 | 8 | 82.12 | 6 | 251.55 |
| TuRBO | 76 | 40 | 568.59 | — | — |

It will be appreciated that, although the various steps in the flowcharts involved in the embodiments as described above are shown in sequence as indicated by the arrows, these steps are not necessarily performed in the order indicated by the arrows. These steps are performed in no strict order unless explicitly stated herein, and these steps may be performed in other orders. Moreover, at least some of the steps in the flowcharts involved in the embodiments as described above may include multiple steps or multiple stages. These steps or stages are not necessarily performed at the same time, but may be performed at different times. These steps or stages are not necessarily performed in sequence, but may be performed in turn or in alternation with other steps or at least some of the steps or stages in other steps.

Based on the same inventive concept, embodiments of this application also provide an object determining apparatus for realizing the object determining method described above. The implementation scheme for solving the problem provided by the apparatus is similar to the implementation scheme described in the foregoing method. Therefore, the specific definition of one or more embodiments of the object determining apparatus provided below may be referred to the definition of the object determining method above, and will not be repeated herein.

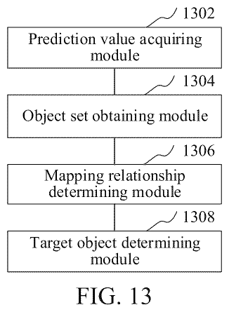

In some embodiments, as shown in FIG. 13, an object determining apparatus is provided, including: a prediction value acquiring module 1302, an object set obtaining module 1304, a mapping relationship determining module 1306, and a target object determining module 1308.

The prediction value acquiring module 1302 is configured to acquire index prediction values of objects in a first object set on a preset index respectively.

The object set obtaining module 1304 is configured to select an object with the index prediction value satisfying index value screening conditions from the first object set to obtain a second object set.

The mapping relationship determining module 1306 is configured to determine, based on index experimental values and object features of the plurality of objects in the first object set on the preset index, a mapping relationship between the preset index and the object features.

The target object determining module 1308 is configured to determine a target object meeting index requirements of the preset index from the second object set based on the mapping relationship.

In some embodiments, the objects in the first object set are mutant proteins. The apparatus further includes: a reference object set screening module, configured to screen based on the first object set to obtain a reference object set. The reference object set satisfies a condition that each amino acid occurs at each mutation position for at least a target frequency. The prediction value acquiring module is further configured to: train an index detection model based on an object feature and an index experimental value of each object in the reference object set; and predict the index prediction value of each object in the first object set by using the trained index detection model.

In some embodiments, the mapping relationship determining module is further configured to: determine the object features of the objects in the reference object set based on the index experimental value of each object in the reference object set on the preset index; and determine a mapping relationship between the preset index and the object features based on the index experimental value and the object feature of each object in the reference object set on the preset index.

In some embodiments, the reference object set screening module is further configured to: acquire a current score set, the current score set including a current score corresponding to each amino acid; obtain a second protein set based on the first object set, and select a target protein from the second protein set based on the current score set; decrease a current score corresponding to an amino acid at each mutation position in the target protein in the current score set, and move the target protein from the second protein set to a first protein set; and reselect, when the current score set characterizes the first protein set as not satisfying the condition that each amino acid occurs at each mutation position for at least the target frequency, a target protein from the second protein set based on the current score set until the current score set characterizes the first protein set as satisfying the condition that each amino acid occurs at each mutation position for at least the target frequency, and determine the first protein set as the reference object set.

In some embodiments, the reference object set screening module is further configured to: acquire an initial score set, an initial score corresponding to each amino acid in the initial score set being the target frequency; and decrease an initial score corresponding to an amino acid at each mutation position in a wild-type protein, respectively, from the initial score set to obtain the current score set, and determine the first protein set based on the wild-type protein, the wild-type protein being an unmutated protein.

In some embodiments, the mapping relationship determining module is further configured to: determine, for each mutant protein in the second protein set, a current score corresponding to an amino acid at each mutation position in the mutant protein, respectively, from the current score set; determine a current protein score of the mutant protein based on the obtained current scores; and select the target protein from the second protein set based on the current protein score.

In some embodiments, each amino acid corresponds to an amino acid, and the score in the current score set is uniquely identified by the amino acid and the mutation position. The mapping relationship determining module is further configured to determine, for the amino acid at each mutation position, a current score corresponding to the amino acid at the mutation position from the current score set according to the amino acid corresponding to the amino acid and the mutation position.

In some embodiments, in response to the object feature being a protein feature, the mapping relationship determining module is further configured to: divide, for each mutation position, the reference object set according to the type of amino acids at the mutation positions to obtain a first sub-object set corresponding to each amino acid; determine, for each amino acid at each mutation position, an amino acid feature of the amino acid at the mutation position based on an index experimental value of each object in the first sub-object set corresponding to the amino acid; and obtain a protein feature of the object based on the amino acid feature of the amino acid at each mutation position in the object.

In some embodiments, the mapping relationship determining module is further configured to: statistically calculate the index experimental value of each object in the first sub-object set corresponding to the amino acid to obtain at least one index experimental statistical value; and determine the amino acid feature of the amino acid at the mutation position based on the at least one index experimental statistical value.

In some embodiments, the mapping relationship determining module is further configured to: perform mean calculation on the index experimental value of each object in the first sub-object set corresponding to the amino acid to obtain a first index mean; and determine a maximum index experimental value to obtain a first index maximum from the index experimental value of each object in the first sub-object set corresponding to the amino acid, the at least one index experimental statistical value including at least one of the first index mean or the first index maximum.

In some embodiments, the mapping relationship determining module is further configured to combine the first index mean and the first index maximum into the amino acid feature of the amino acid at the mutation position.

In some embodiments, in response to the object feature being a protein feature, the mapping relationship determining module is further configured to: determine, for each amino acid, an object in which an amino acid at a mutation position includes the amino acid from the reference object set to obtain a second sub-object set corresponding to the amino acid; determine, for each amino acid, an amino acid feature of the amino acid based on index experimental values of objects in the second sub-object set corresponding to the amino acid; and obtain a protein feature of the object based on the amino acid feature of the amino acid at each mutation position in the object.

In some embodiments, the target object determining module is further configured to: determine a statistical index value of each object in the second object set on a target statistical index based on the mapping relationship, and determine a selected object from the second object set based on the statistical index value; add the selected object to the reference object set when iteration stop conditions are not satisfied; redetermine the object features of the objects in the reference object set based on the index experimental value of each object in the reference object set on the preset index until the iteration stop conditions are satisfied; and determine the selected object obtained when the iteration stop conditions are satisfied as the target object meeting the index requirements of the preset index.

The modules in the object determining apparatus may be implemented in whole or in part by software, hardware, and combinations thereof. The modules may be built in or independent of a processor of a computer device in a hardware form, or may be stored in a memory of the computer device in a software form, so that the processor invokes and performs an operation corresponding to each of the foregoing modules.

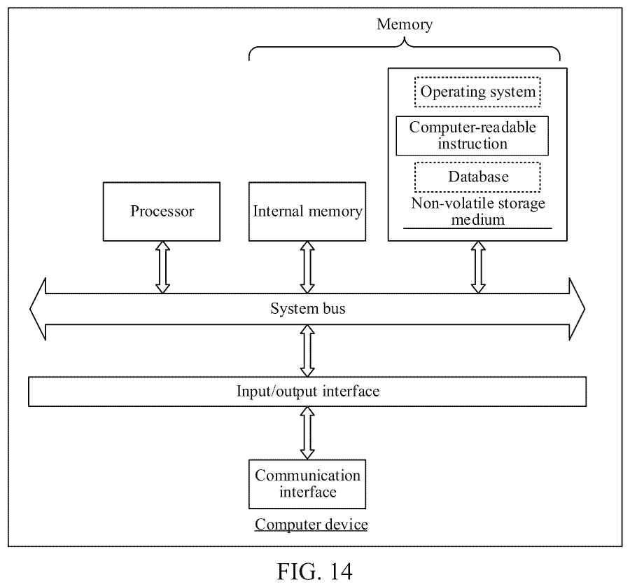

In some embodiments, a computer device is provided. The computer device may be a server, and an internal structure diagram thereof may be shown in FIG. 14. The computer device includes a processor, a memory, an input/output (I/O) interface, and a communication interface. The processor, the memory, and the I/O interface are connected via a system bus. The communication interface is connected to the system bus via the I/O interface. The processor of the computer device is configured to provide computing and control capabilities. The memory of the computer device includes a non-transitory storage medium and an internal memory. The non-transitory storage medium stores an operating system, computer-readable instructions, and a database. The internal memory provides an environment for running of the operating system and the computer-readable instructions in the non-transitory storage medium. The database of the computer device is configured to store data involved in the object determining method. The I/O interface of the computer device is configured to exchange information between the processor and an external device. The communication interface of the computer device is configured to communicate with an external terminal through a network connection. The computer-readable instruction is executed by the processor to implement an object determining method.

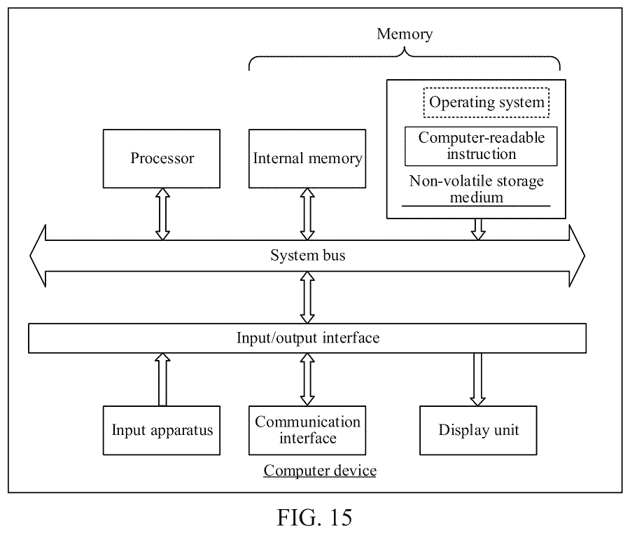

In some embodiments, a computer device is provided. The computer device may be a terminal, and an internal structure diagram thereof may be shown in FIG. 15. The computer device includes a processor, a memory, an I/O interface, a communication interface, a display unit, and an input apparatus. The processor, the memory, and the I/O interface are connected via a system bus. The communication interface, the display unit, and the input apparatus are connected to a system bus via the I/O interface. The processor of the computer device is configured to provide computing and control capabilities. The memory of the computer device includes a non-transitory storage medium and an internal memory. The non-transitory storage medium stores an operating system and computer-readable instructions. The internal memory provides an environment for running of the operating system and the computer-readable instructions in the non-transitory storage medium. The I/O interface of the computer device is configured to exchange information between the processor and an external device. The communication interface of the computer device is configured for wired or wireless communication with an external terminal. The wireless communication may be realized through WIFI, a mobile cellular network, near-field communication (NFC), or other technologies. The computer-readable instruction is executed by the processor to implement an object determining method. The display unit of the computer device is configured to form a visually visible picture, and may be a display screen, a projection apparatus, or a virtual reality imaging apparatus. The display screen may be a liquid crystal display screen or an electronic ink display screen. The input apparatus of the computer device may be a touch layer covering the display screen, or may be a key, a trackball, or a touch pad disposed on a housing of the computer device, or may be an external keyboard, a touch pad, a mouse, or the like.

It will be appreciated by a person skilled in the art that the structures shown in FIGS. 14 and 15 are merely block diagrams of some of the structures relevant to the solution of this application and do not constitute a limitation of the computer device to which the solution of this application is applied. The specific computer device may include more or less components than those shown in the figures, or include some components combined, or have different component arrangements.

In some embodiments, a computer device is provided, which includes a memory and one or more processors. The memory stores computer-readable instructions. The computer-readable instructions, when executed by the processor, enable the one or more processors to perform the steps of the object determining method.

In some embodiments, one or more non-transitory readable storage media are provided, which store computer-readable instructions. The computer-readable instructions, when executed by one or more processors, enable the one or more processors to implement the steps of the object determining method.

In some embodiments, a computer program product is provided, which includes computer-readable instructions. The computer-readable instructions, when executed by a processor, implement the steps of the object determining method.

User information (including but not limited to user equipment information, user personal information, and the like) and data (including but not limited to data used for analysis, stored data, displayed data, and the like) involved in this application are information and data authorized by users or fully authorized by all parties, and the collection, use and processing of relevant data shall comply with relevant laws, regulations and standards of relevant countries and regions.

It will be appreciated by a person of ordinary skill in the art that implementing all or part of the processes in the foregoing method embodiments may be accomplished by instructing associated hardware through computer-readable instructions. The computer-readable instructions may be stored on a non-transitory computer-readable storage medium. The computer-readable instructions, when executed, may include the processes in the foregoing method embodiments. Any reference to a memory, a database, or another medium used in the various embodiments provided by this application may include at least one of non-volatile and volatile memories. The non-volatile memory may include a read-only memory (ROM), a magnetic tape, a floppy disk, a flash memory, an optical memory, a high-density embedded non-volatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM) or an external cache. For the purpose of description instead of limitation, the RAM is available in a plurality of forms, such as a static random access memory (SRAM) or a dynamic random access memory (DRAM). The database involved in the embodiments provided by this application may include at least one of a relational database and a non-relational database. The non-relational database may include, but is not limited to, a blockchain-based distributed database. The processor in the embodiments provided by this application may include, but is not limited to, a general purpose processor, a central processing unit, a graphics processor, a digital signal processor, a programmable logic device, and a quantum computing-based data processing logic unit.

The technical features of the foregoing embodiments may be combined in any combination. In order to make the description concise, not all the possible combinations of the technical features in the foregoing embodiments are described. However, as long as there is no contradiction between the combinations of these technical features, the combinations are to be considered within the scope of this specification. In this application, the term “module” in this application refers to a computer program or part of the computer program that has a predefined function and works together with other related parts to achieve a predefined goal and may be all or partially implemented by using software, hardware (e.g., processing circuitry and/or memory configured to perform the predefined functions), or a combination thereof. Each module can be implemented using one or more processors (or processors and memory). Likewise, a processor (or processors and memory) can be used to implement one or more modules. Moreover, each module can be part of an overall module that includes the functionalities of the module.

The foregoing embodiments only describe several implementations of this application, which are described specifically and in detail, but cannot be construed as a limitation to the patent scope of this application. It will be appreciated by a person of ordinary skill in the art that several transformations and improvements may be made without departing from the concept of this application. These transformations and improvements belong to the protection scope of this application. Therefore, the protection scope of this application shall be subject to the appended claims.

What is claimed is:

1. An object determining method, performed by a computer device, the method comprising:
acquiring index prediction values of objects in a first object set on a preset index respectively;
determining, based on index experimental values and object features of the objects in the first object set on the preset index, a mapping relationship between the preset index and the object features;
selecting, from the first object set, objects with the index prediction values satisfying index value screening conditions, to obtain a second object set; and
determining a target object meeting index requirements of the preset index from the second object set based on the mapping relationship.

2. The method according to claim 1, wherein the objects in the first object set are mutant proteins, and the method further comprises:
screening based on the first object set to obtain a reference object set, the reference object set satisfying a condition that each amino acid occurs at each mutation position for at least a target frequency; and
training an index detection model based on an object feature and an index experimental value of each object in the reference object set; and
predicting the index prediction value of each object in the first object set by using the trained index detection model.

3. The method according to claim 2, wherein the determining, based on index experimental values and object features of the objects in the first object set on the preset index, a mapping relationship between the preset index and the object features comprises:
determining the object features of the objects in the reference object set based on the index experimental values of the objects in the reference object set on the preset index; and
determining a mapping relationship between the preset index and the object features based on the index experimental values and the object features of the objects in the reference object set on the preset index.

4. The method according to claim 2, wherein the screening based on the first object set to obtain a reference object set comprises:
acquiring a current score set, the current score set comprising a current score corresponding to each amino acid;
obtaining a second protein set based on the first object set, and selecting a target protein from the second protein set based on the current score set;
decreasing a current score corresponding to an amino acid at each mutation position in the target protein in the current score set, and moving the target protein from the second protein set to a first protein set; and
reselecting, when the current score set characterizes the first protein set as not satisfying the condition that each amino acid occurs at each mutation position for at least the target frequency, a target protein from the second protein set based on the current score set until the current score set characterizes the first protein set as satisfying the condition that each amino acid occurs at each mutation position for at least the target frequency, and determining the first protein set as the reference object set.

5. The method according to claim 4, wherein the acquiring a current score set comprises:
acquiring an initial score set, an initial score corresponding to each amino acid in the initial score set being the target frequency; and
decreasing an initial score corresponding to an amino acid at each mutation position in a wild-type protein, respectively, from the initial score set to obtain the current score set, and determining the first protein set based on the wild-type protein, the wild-type protein being an unmutated protein.

6. The method according to claim 4, wherein the selecting a target protein from the second protein set based on the current score set comprises:
determining, for each mutant protein in the second protein set, a current score corresponding to an amino acid at each mutation position in the mutant protein, respectively, from the current score set;
determining a current protein score of the mutant protein based on the obtained current scores; and
selecting the target protein from the second protein set based on the current protein score.

7. The method according to claim 6, wherein each amino acid corresponds to an amino acid, and the score in the current score set is uniquely identified by the amino acid and the mutation position; and
the determining a current score corresponding to an amino acid at each mutation position in the mutant protein, respectively, from the current score set comprises:
determining, for the amino acid at each mutation position, a current score corresponding to the amino acid at the mutation position from the current score set according to the amino acid corresponding to the amino acid and the mutation position.

8. The method according to claim 3, wherein in response to the object feature being a protein feature, the determining the object features of the objects in the reference object set based on the index experimental value of each object in the reference object set on the preset index comprises:
dividing, for each mutation position, the reference object set according to the type of amino acids at the mutation positions to obtain a first sub-object set corresponding to each amino acid;
determining, for each amino acid at each mutation position, an amino acid feature of the amino acid at the mutation position based on an index experimental value of each object in the first sub-object set corresponding to the amino acid; and
obtaining a protein feature of the object based on the amino acid feature of the amino acid at each mutation position in the object.

9. The method according to claim 8, wherein the determining an amino acid feature of the amino acid at the mutation position based on an index experimental value of each object in the first sub-object set corresponding to the amino acid comprises:
statistically calculating the index experimental value of each object in the first sub-object set corresponding to the amino acid to obtain at least one index experimental statistical value; and
determining the amino acid feature of the amino acid at the mutation position based on the at least one index experimental statistical value.

10. The method according to claim 9, wherein the statistically calculating the index experimental value of each object in the first sub-object set corresponding to the amino acid to obtain at least one index experimental statistical value comprises:
performing mean calculation on the index experimental value of each object in the first sub-object set corresponding to the amino acid to obtain a first index mean; and
determining a maximum index experimental value to obtain a first index maximum from the index experimental value of each object in the first sub-object set corresponding to the amino acid, the at least one index experimental statistical value comprising at least one of the first index mean or the first index maximum.

11. The method according to claim 10, wherein the determining the amino acid feature of the amino acid at the mutation position based on the at least one index experimental statistical value comprises:
combining the first index mean and the first index maximum into the amino acid feature of the amino acid at the mutation position.

12. The method according to claim 3, wherein in response to the object feature being a protein feature, the determining the object features of the objects in the reference object set based on the index experimental value of each object in the reference object set on the preset index comprises:
determining, for each amino acid, an object in which an amino acid at a mutation position comprises the amino acid from the reference object set to obtain a second sub-object set corresponding to the amino acid;
determining, for each amino acid, an amino acid feature of the amino acid based on index experimental values of objects in the second sub-object set corresponding to the amino acid; and
obtaining a protein feature of the object based on the amino acid feature of the amino acid at each mutation position in the object.

13. The method according to claim 3, wherein the determining a target object meeting index requirements of the preset index from the second object set based on the mapping relationship comprises:
determining a statistical index value of each object in the second object set on a target statistical index based on the mapping relationship, and determining a selected object from the second object set based on the statistical index value;
adding the selected object to the reference object set when iteration stop conditions are not satisfied;
redetermining the object features of the objects in the reference object set based on the index experimental value of each object in the reference object set on the preset index until the iteration stop conditions are satisfied; and
determining the selected object obtained when the iteration stop conditions are satisfied as the target object meeting the index requirements of the preset index.

14. A computer device, comprising a memory and one or more processors, the memory storing computer-readable instructions, the computer-readable instructions, when executed by the processor, enabling the computer device to perform an object determining method including:
acquiring index prediction values of objects in a first object set on a preset index respectively;
determining, based on index experimental values and object features of the objects in the first object set on the preset index, a mapping relationship between the preset index and the object features;
selecting, from the first object set, objects with the index prediction values satisfying index value screening conditions, to obtain a second object set; and
determining a target object meeting index requirements of the preset index from the second object set based on the mapping relationship.

15. The computer device according to claim 14, wherein the objects in the first object set are mutant proteins, and the method further comprises:
screening based on the first object set to obtain a reference object set, the reference object set satisfying a condition that each amino acid occurs at each mutation position for at least a target frequency; and
training an index detection model based on an object feature and an index experimental value of each object in the reference object set; and
predicting the index prediction value of each object in the first object set by using the trained index detection model.

16. The computer device according to claim 15, wherein the determining, based on index experimental values and object features of the objects in the first object set on the preset index, a mapping relationship between the preset index and the object features comprises:
determining the object features of the objects in the reference object set based on the index experimental values of the objects in the reference object set on the preset index; and
determining a mapping relationship between the preset index and the object features based on the index experimental values and the object features of the objects in the reference object set on the preset index.

17. The computer device according to claim 15, wherein the screening based on the first object set to obtain a reference object set comprises:
acquiring a current score set, the current score set comprising a current score corresponding to each amino acid;
obtaining a second protein set based on the first object set, and selecting a target protein from the second protein set based on the current score set;
decreasing a current score corresponding to an amino acid at each mutation position in the target protein in the current score set, and moving the target protein from the second protein set to a first protein set; and
reselecting, when the current score set characterizes the first protein set as not satisfying the condition that each amino acid occurs at each mutation position for at least the target frequency, a target protein from the second protein set based on the current score set until the current score set characterizes the first protein set as satisfying the condition that each amino acid occurs at each mutation position for at least the target frequency, and determining the first protein set as the reference object set.

18. One or more non-transitory readable storage media, storing computer-readable instructions, the computer-readable instructions, when executed by one or more processors of a computer device, enabling the computer device to perform an object determining method including:
acquiring index prediction values of objects in a first object set on a preset index respectively;
determining, based on index experimental values and object features of the objects in the first object set on the preset index, a mapping relationship between the preset index and the object features;
selecting, from the first object set, objects with the index prediction values satisfying index value screening conditions, to obtain a second object set; and
determining a target object meeting index requirements of the preset index from the second object set based on the mapping relationship.

19. The non-transitory readable storage media according to claim 18, wherein the objects in the first object set are mutant proteins, and the method further comprises:
screening based on the first object set to obtain a reference object set, the reference object set satisfying a condition that each amino acid occurs at each mutation position for at least a target frequency; and
training an index detection model based on an object feature and an index experimental value of each object in the reference object set; and
predicting the index prediction value of each object in the first object set by using the trained index detection model.

20. The non-transitory readable storage media according to claim 19, wherein the determining, based on index experimental values and object features of the objects in the first object set on the preset index, a mapping relationship between the preset index and the object features comprises:
determining the object features of the objects in the reference object set based on the index experimental values of the objects in the reference object set on the preset index; and
determining a mapping relationship between the preset index and the object features based on the index experimental values and the object features of the objects in the reference object set on the preset index.
